(19) United States
(12) Patent Application Publication
(10) Pub. No.: US 2025/0060368 A1
(43) Pub. Date: Feb. 20, 2025

(54) METHODS OF DETECTING AND TREATING IMMUNOTHERAPY-RESISTANT CANCER

(71) Applicants: Washington University, St. Louis, MO (US); The Trustees of the University of Pennsylvania, Philadelphia, PA (US); University of Augsburg, Augsburg (DE)

(72) Inventors: Nathan Singh, St. Louis, MO (US); Armin Ghobadi, St. Louis, MO (US); Marco Ruella, Philadelphia, PA (US); Saar Gill, Philadelphia, PA (US); Regina Fluhrer, Augsburg (DE)

(73) Assignees: Washington University, St. Louis, MO (US); The Trustees of the University of Pennsylvania, Philadelphia, PA (US); University of Augsburg, Augsburg, PA (DE)

(21) Appl. No.: 18/165,683

(22) Filed: Feb. 7, 2023

Related U.S. Application Data
(60) Provisional application No. 63/307,228, filed on Feb. 7, 2022.

(51) Int. Cl. G01N 33/574

(52) U.S. Cl. CPC G01N 33/57492; G01N 33/57426; G01N 2333/70503; G01N 2440/38

(57) ABSTRACT

Among the various aspects of the present disclosure is the provision of methods of detecting and treating immunotherapy-resistant B cell malignancies. Methods of detection include detecting a glycosylation state of CD19 or detecting expression or activity of SPPL3 in malignant B cells, comparing to a reference state or value, and determining that the B cell malignancy is resistant to immunotherapy if the glycosylation state of CD19 or expression or activity of SPPL3 is substantially increased or decreased relative to corresponding reference levels. Also provided are therapeutic agents capable of modulating CD19 glycosylation or SPPL3 expression or activity for treatment of B cell malignancies in combination with CD19-targeted immunotherapies.

### CROSS-REFERENCE TO RELATED APPLICATIONS

This application claims the benefit of priority to U.S. Provisional Application No. 63/307,228 filed on Feb. 7, 2022, the content of which is incorporated by reference in its entirety.

### STATEMENT REGARDING FEDERALLY SPONSORED RESEARCH OR DEVELOPMENT

This invention was made with government support under CA212302 and CA194256 awarded by the National Institutes of Health. The government has certain rights in the invention.

### MATERIAL INCORPORATED-BY-REFERENCE

Not applicable.

### FIELD

The present disclosure generally relates to compositions and methods for detecting or treating immunotherapy-resistant cancers.

### SUMMARY

Among the various aspects of the present disclosure is the provision of compositions and methods of detecting and treating immunotherapy-resistant B cell malignancies. The disclosed compositions and methods are based on the discovery that recurrent, refractory, or therapy-resistant B cell malignancies are characterized, in at least some instances, by B cells with CD19 markers that are hyperglycosylated or hypoglycosylated relative to a reference CD19 glycosylation.

In one aspect, a method of detecting resistance of a B cell malignancy to an immunotherapy in a subject is disclosed that includes detecting a biomarker level in malignant B cells obtained from the subject, the biomarkers selected from a glycosylation state of CD19, an expression or activity of SPPL3, and any combination thereof; comparing the biomarker level detected in the malignant B cells to a reference biomarker level; and determining that the B cell malignancy is resistant to immunotherapy if the biomarker level is substantially different from the reference biomarker level. In some aspects, determining that the B cell malignancy is resistant to immunotherapy further includes determining that the B cell malignancy is resistant to immunotherapy if the glycosylation state of CD19 is substantially hyperglycosylated or hypoglycosylated compared to the reference glycosylation state in the malignant B cells, the expression or activity of SPPL3 is substantially increased or decreased compared to the reference expression or activity of SPPL3 in the malignant B cells, any combination thereof. In some aspects, the malignant B cells obtained from the subject include a mutation affecting CD19 glycosylation. In some aspects, the mutation affecting CD19 glycosylation is a CD19ΔTyr260 mutation. In some aspects, the malignant B cells obtained from the subject appear negative for CD19 expression as detected via flow cytometry or anti-CD19 antibodies configured to recognize CD19 comprising the reference glycosylation state.

In another aspect, a method of treating a B cell malignancy in a subject is disclosed that includes administering a treatment to the subject comprising one of administering to the subject a therapeutically effective amount of a CD19-targeted immunotherapy in combination with a therapeutically effective amount of an agent capable of modulating CD19 glycosylation or SPPL3 expression or activity in malignant B cells, or administering to the subject a therapeutically effective amount of a modified CD19-targeted immunotherapy comprising an antibody or a CAR T cell that specifically binds hyperglycosylated or hypoglycosylated CD19 of malignant B cells. In some aspects, administering the treatment improves T cell recognition of CD19, increases activation of CAR T cell effector function, or enhances CD19-targeted CAR T anti-tumor cytotoxicity. In some aspects, the CD19-targeted immunotherapy includes a CD19-targeted CAR T cell therapy or a CD19-targeted bispecific T cell engager. In some aspects, the B cell malignancy includes a lymphoma or leukemia. In some aspects, the B cell malignancy is recurrent, refractory, or resistant to immunotherapy.

In an additional aspect, a method of selecting a treatment for a B cell malignancy in a subject is disclosed that includes detecting a biomarker level of CD19 in malignant B cells obtained from the subject, the biomarkers selected from a glycosylation state of CD19, an expression or activity of SPPL3, and any combination thereof, comparing the biomarker level detected in the malignant B cells to a reference biomarker level, selecting a treatment for the B cell malignancy based on the comparison of the biomarker level to the reference biomarker level. In some aspects, selecting the treatment for the B cell malignancy further includes selecting a CD19-targeted immunotherapy for the treatment if the glycosylation state of CD19 is similar to the reference glycosylation state, the expression or activity of SPPL3 is similar to the reference expression or activity of SPPL3, and any combination thereof; selecting one of the CD19-targeted immunotherapy in combination with an agent capable of modulating CD19 glycosylation, or a modified CD19-targeted immunotherapy comprising an antibody, a bispecific T cell engager, or a CAR T cell that specifically binds hyperglycosylated or hypoglycosylated CD19 of malignant B cells if the glycosylation state of CD19 is substantially hyperglycosylated or hypoglycosylated compared to the reference glycosylation state; or selecting for the treatment the CD19-targeted immunotherapy in combination with an agent capable of modulating expression or activity of SPPL3 if the expression or activity of SPPL3 is substantially increased or decreased compared to the reference expression or activity of SPPL3. In some aspects, administering the treatment improves T cell recognition of CD19, increases activation of CAR T cell effector function, or enhances CD19-targeted CAR T anti-tumor cytotoxicity. In some aspects, the CD19-targeted immunotherapy includes a CD19-targeted CAR T cell therapy or a CD19-targeted bispecific T cell engager. In some aspects, the B cell malignancy includes a lymphoma or leukemia. In some aspects, the B cell malignancy is recurrent, refractory, or resistant to immunotherapy. In some aspects, the malignant B cells obtained from the subject include a mutation affecting CD19 glycosylation. In some aspects, the mutation affecting CD19 glycosylation is a CD19ΔTyr260 mutation. In some aspects, the malignant B cells obtained from the subject appear negative for CD19 expression as detected via flow cytometry or anti-CD19 antibodies configured to recognize CD19 comprising the reference glycosylation state.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic diagram illustrating a genome-wide knockout screen in Nalm6 ALL described herein.

FIG. 1B is a plot of MAGeCK beta scores for genes that were enriched in CART19 targeted cells compared to untargeted T cells.

FIG. 1C is a survival curve of Nalm6 48 hours after combination with CART19 (E:T ratio 0.25:1). ****P<0.0001 by two-way ANOVA with Bonferroni correction for multiple comparisons. Data are representative of n=5 individual experiments with distinct donor T cells. Statistical annotations reflect differences between WT and SPPL3KO clone 1 (upper) and WT and SPPL3KO clone 2 (lower).

FIG. 1D is a survival curve of Nalm6 over time after combination with CART19 (E:T ratio 0.25:1). ****P<0.0001 by two-way ANOVA with Bonferroni correction for multiple comparisons. Data are representative of n=5 individual experiments with distinct donor T cells. Statistical annotations reflect differences between WT and SPPL3KO clone 1 (upper) and WT and SPPL3KO clone 2 (lower).

FIG. 1E is a survival curve of OCI-Ly10 48 hours after combination with CART19 (E:T 0.25:1). ****P<0.0001 by two-way ANOVA with Bonferroni correction for multiple comparisons. Data are representative of n=3 individual experiments with distinct donor T cells.

FIG. 1F is a survival curve of OCI-Ly10 over time after combination with CART19 (E:T 0.25:1). ***P<0.001, ****P<0.0001 by two-way ANOVA with Bonferroni correction for multiple comparisons. Data are representative of n=3 individual experiments with distinct donor T cells.

FIG. 2A is a schematic diagram illustrating an experimental scheme used for T cell activation studies described herein.

FIG. 2B is a graph of the expression of CD69 on T cells after overnight co-culture with WT or SPPL3KO Nalm6 cells or alone (control).

FIG. 2C is a graph of the expression of PD1 on T cells after overnight co-culture with WT or SPPL3KO Nalm6 cells or alone (control).

FIG. 2D is a graph of the expression of Tim3 on T cells after overnight co-culture with WT or SPPL3KO Nalm6 cells or alone (control).

FIG. 2E is a graph of the expression of CD107a on T cells after a four-hour co-culture with WT or SPPL3KO Nalm6 cells.

FIG. 2F is a graph of the expression of GFP reflecting the binding of NFAT and NFκB to their respective promoter sites in CD19 CAR-expressing Jurkat cells.

FIG. 2G is a graph of the expression of eCFP, reflecting the binding of NFAT and NFκB to their respective promoter sites in CD19 CAR-expressing Jurkat cells.

FIG. 2H is a schematic diagram illustrating a scheme used for a long-term co-culture and re-challenge study described herein.

FIG. 2I is a survival of WT Nalm6 over time after the combination of CART19 cells previously cultured with either WT or SPPL3KO Nalm6 cells. Representative of n=2 individual experiments with distinct donor T cells. **** P<0.0001 by two-way ANOVA with Bonferroni correction for multiple comparisons.

FIG. 3A shows Western blot assays of lysates from WT or SPPL3KO Nalm6 and OCI-Ly10 cells probed for CD19. Protein electrophoresis was performed on a 15% polyacrylamide gel. Representative of n=4 individual experiments.

FIG. 3B shows Western blot assays of lysates from WT or SPPL3KO Nalm6 cells that were either untreated (UT), treated with PNGase F (PNG), or treated with Endoglycosidase H (Endo) and then probed for CD19. Representative data of n=3 individual experiments. Electrophoresis was performed on a 6% polyacrylamide gel.

FIG. 3C is a graph of the Median Fluorescence Intensity of CD19 as detected by FMC63-APC on the surface of WT or SPPL3KO Nalm6 cells.

FIG. 3D is a ribbon diagram illustrating the structural modeling of the FMC63 binding epitope on CD19. Residues in blue (R144, W140, and P203) are essential for antibody binding, while residues in red (N125 and N114) are potentially N-glycosylated.

FIG. 3E shows Western blot assays of lysates from WT or SPPL3KO Nalm6 cells probed for CD22. Representative data from n=2 individual experiments.

FIG. 3F is a survival curve of WT or SPPL3KO Nalm6 cells 48 hours after combination with CART22 (E:T ratio 0.25:1). Representative data from n=3 individual experiments with distinct donor T cells. ****P<0.0001 by two-way ANOVA with Bonferroni correction for multiple comparisons.

FIG. 3G is a survival curve of WT or SPPL3KO Nalm6 cells over time after combination with CART22 (E:T ratio 0.25:1). Representative data from n=3 individual experiments with distinct donor T cells. ****P<0.0001 by two-way ANOVA with Bonferroni correction for multiple comparisons.

FIG. 4A shows Western blot assays of lysates from WT, SPPL3KO or SPPL3KO with over-expression of SPPL3 (SPPL3KO+) Nalm6 cells probed for CD19 and SPPL3.

FIG. 4B shows a sequential flow cytometric evaluation of FMC63-APC binding on SPPL3KO+Nalm6 after over-expression of SPPL3.

FIG. 4C is a survival curve of WT, SPPL3KO or SPPL3KO+Nalm6 over time when established on day 10 after SPPL3 re-introduction (E:T 0.25:1). **** P<0.0001 by two-way ANOVA with Bonferroni correction for multiple comparisons. Statistical annotations reflect differences between WT and SPPL3KO (upper) or WT and SPPL3KO+Nalm6 (lower).

FIG. 4D is a survival curve of WT, SPPL3KO or SPPL3KO+Nalm6 over time when established on day 20 after re-introduction of SPPL3 (E:T 0.25:1). ****P<0.0001 by two-way ANOVA with Bonferroni correction for multiple comparisons. Statistical annotations reflect differences between WT and SPPL3KO (upper) or WT and SPPL3KO+Nalm6 (lower).

FIG. 5 is a schematic diagram illustrating a proposed model for a mechanism of glycosylation-mediated antigen escape.

FIG. 6A is a schematic diagram illustrating gene domains of wild-type and Tyr260 mutant CD19.

FIG. 6B is a schematic diagram illustrating an inferred evolutionary trajectory starting from a CD19-positive acute lymphoblastic leukemia (ALL) and developing into CD19 mutant (CD19ΔTyr260) ALL.

FIG. 6C is a set of graphs summarizing levels of mRNA expression of genes of interest in baseline, post-blinatumomab/pre-CAR T-cell therapy, and post-CAR T-cell therapy samples. Values represent reads per kilobase per million mapped reads (RPKM). Gene expression of class II transactivator (CIITA) and major histocompatibility complex class II genes are compared with baseline. TM, transmembrane.

FIG. 7A is a schematic diagram illustrating wild-type (wt) and Tyr260 mutant (mut) lentiviral expression vectors transduced into the CD19 negative Jurkat cells.

FIG. 7B is a set of plots from a flow cytometry assay of wt and Tyr260 mutant samples using anti-CD19 antibody clones, H1B19 and SJ25C1.

FIG. 7C shows Western blot assays of whole cell lysate (WCL), cytoplasmic fraction (CIB), and membrane fraction (MIB) of non-transduced (neg) and transduced Jurkat cells.

FIG. 7D shows Western blot assays of neg and transduced Jurkat cells treated with and without peptide N glycosidase (PNGase).

FIG. 7E is a graph summarizing CART19 killing assay measurements of neg and transduced Jurkat cells. Bars represent the mean (±SD) percentage of the remaining GFP target normalized to tumor-only controls (n=3 for CD19 negative control; n=4 for CD19 wt and CD19 mut).

FIG. 7F is a graph summarizing % GFP+ and GFP-tumor cells after 19-28ζ CAR T treatments; 19-28ζ CAR T selectively killed CD19 wt Jurkat (GFP+/cell trace violet+), but not neg control Jurkat (GFP−/cell trace violet+) when mixed at a 1:1 ratio in flow-based killing assays. Bars represent the mean (±SD) percent of GFP negative and GFP negative as a proportion of total assayed Jurkat cells for each experimental condition (n=4).

FIG. 7G is a pair of representative fluorescence-activated single-cell sorting plots of transduced Jurkat cells following an 18-hour killing assay.

FIG. 7H contains schematic and ribbon diagrams illustrating in silico modeling of a 3D structure of wild-type CD19. CTV, and cell trace violet.

FIG. 7I contains schematic and ribbon diagrams illustrating in silico modeling of CD19ΔTyr260. CTV, cell trace violet.

Those of skill in the art will understand that the drawings, described below, are for illustrative purposes only. The drawings are not intended to limit the scope of the present teachings in any way.

### DETAILED DESCRIPTION

The present disclosure is based, at least in part, on the discovery that SPPL3 in malignant B cells regulates glycosylation of CD19 and resistance to immunotherapy. As shown herein, altered SPPL3 expression or glycosylation of CD19 leads to antigen-mediated tumor escape from CAR T cell therapy.

CD19-targeting immunotherapies have revolutionized the care of patients with CD19+B cell cancers. Many patients are ineligible for therapy because their cancer cells lack CD19 expression. Other patients who do receive therapy fail therapy because their cancer cells lose CD19 expression. As described herein, it was discovered that alterations in CD19 glycosylation, a process in which sugar moieties are added to the CD19 protein, can both impair the ability to detect CD19 using traditional diagnostic techniques and impair immunotherapies from detecting CD19 on cancer cells. Also described herein is a protein, SPPL3, which indirectly regulates CD19 glycosylation and thereby can directly control response to CD19-targeted T cell therapies. Also described herein are genetic variants including, but not limited to, a CD19ΔTyr260 mutation, which indirectly regulates CD19 glycosylation and thereby can directly control response to CD19-targeted T cell therapies.

The altered diagnostic approach to determine CD19 expression described herein enables development of strategies to overcome alterations in glycosylation that will evade this mechanism of therapeutic failure.

Described herein is a method to detect a novel antigen escape mechanism among malignant B cells that are subjected to CD19-directed CAR-T therapy. It was discovered that a major cause of B cell lymphoma resistance to CAR-T and antibody therapy is via changes in CD19 glycosylation. Two core CD19-related mutations are described herein: A) spontaneous deletion of Tyr260 on the CD19 protein, which results in alterations in CD19 secondary structure, glycosylation, and tumor resistance to FDA-approved CAR-T therapy (Kymriah and Yescarta), and B) changes in the expression/activity of SPPL3, which broadly affects glycosylation of a variety of plasma membrane proteins, including CD19, and indirectly confers tumor resistance to FDA-approved CAR-T therapies.

As described herein, SPPL3 deletion was a top hit in a genome-wide screen to discover loss-of-function mutations contributing to tumor resistance against anti-CD19 therapies. Deletion of SPPL3 leads to CD19 hyperglycosylation, whereas overexpression of SPPL3 leads to CD19 hypoglycosylation. The overall expression level, as well as localization of CD19, is otherwise unchanged. Deviations from normal CD19 glycosylation also reduce the detection of this tumor marker by common diagnostic antibodies, such as clones FMC63 and HIB19. However, CD22 detection and response to anti-CD22 CAR-T therapy were not affected by SPPL3 mutations.

Anti-CD19 CAR-T is a promising therapy for B cell malignancies, but total remission is still rare and relapse rates remain frustratingly high. This is in part due to the gradual loss of recognition of tumor CD19 by the effector T cells, commonly called immune escape or antigen escape. Described herein is a fundamental mechanism for antigen escape in CD19-directed antibody or CAR-T therapies, the alteration of CD19 glycosylation.

The methods described herein can be used to detect the emergence of CAR-T resistance by detecting changes in the glycosylation of CD19 or the expression of glycosylation-modulating genes, such as SPPL3. The present disclosure can also be useful in the clinical development of novel CD19 CAR-T therapeutics by factoring in the glycosylation state of the antigen and adjusting the CAR-T scFv for maximum antigen recognition.

Currently, CD19 antibodies are used to detect this marker on B cell cancers, and the failure of these antibodies to detect any significant CD19 signal had been attributed to low tumor expression of CD19 and the unsuitability of the tumors for anti-CD19 therapy. Demonstrated herein is that CD19 is likely expressed normally but exhibits abnormal glycosylation to evade antigen detection. Therefore, anti-CD19 antibodies that recognize different glycosylation states of CD19, such as normal glycosylation, hyperglycosylation, hypoglycosylation, or ΔTyr260-associated glycosylation may be useful in treating cancers resistant to CD19-targeted immunotherapy.

### CD19-Targeted Immunotherapy-Resistant Disease Treatments

As described herein, alterations of CD19 glycosylation have been observed to develop over courses of treatments of various diseases, disorders, and conditions. Further, such alterations of CD19 glycosylation are associated with the development of resistance to various CD19-targeted immunotherapies, as described herein. Abnormal CD19 glycosylation disrupts epitope recognition by antibodies or other antigen-binding domains used to detect and/or target disease cells for treatment.

Without being limited to any particular theory, at least two strategies may be used to treat various diseases, disorders, and conditions using a CD19-targeted immunotherapy agent in a patient exhibiting immunotherapy resistance. In some aspects, the treatment may include administering a glycosylation-modulating compound configured to modulate CD19 glycosylation as an adjuvant in combination with a CD19-CAR T cell therapy for treatment of such conditions. In other aspects, the treatment may include administering a CD19-targeted immunotherapy configured to enable epitope recognition of CD19 with one or more glycosylation profiles associated with refractive or recurring B cell malignancies.

### CD19 Glycolysation-Modulating Compounds

As described herein, CD19 glycosylation-modulating compounds may be configured to upregulate or downregulate CD19 glycosylation as needed to revert the CD19 glycosylation profile of the disease cells to a reference CD19 glycosylation profile. The reference CD19 glycosylation profile, as used herein, refers to a CD19 glycosylation profile that results in enhanced epitope recognition by antibodies or other antigen-binding domains used to detect and/or target disease cells for treatments such as CD19 CAR T cell therapies. By way of a non-limiting example, one reference CD19 glycosylation profile includes a wild-type glycosylation profile corresponding to a wild-type B cell. In various aspects, the reference CD19 glycosylation profile comprises the glycosylation profile of the CD19 epitope corresponding to the targeting antibodies or other targeting moieties of an existing CD19-targeted immunotherapy or any other standard CD19 glycosylation profile without limitation.

In various aspects, CD19 glycosylation-modifying agents include any composition or method that can modulate the degree of glycosylation or glycosylation profile of CD19 on disease cells without limitation. For example, a CD19 glycosylation-modifying agent can be an activator, an inhibitor, an agonist, or an antagonist. As another example, the CD19 glycosylation modulation can be the result of gene editing.

By way of a non-limiting example, the CD19 glycosylation-modifying agent may be a compound configured to modulate SPPL3 expression or activity in disease cells. As described herein, SPPL3 activity or expression has been implicated in various diseases, disorders, and conditions. As such, modulation of SPPL3 activity or expression can be used for treatment of such conditions. In various aspects, SPPL3 modulation can comprise modulating the activity or expression of SPPL3 in cells, modulating the quantity of cells that express SPPL3, or modulating the quality of the SPPL3-expressing cells. In some embodiments, the SPPL3 modulation agent decreases or increases SPPL3 activity or expression, or restores SPPL3 activity or expression to a normal or reference value.

SPPL3 modulation agents can be any composition or method that can modulate SPPL3 expression or activity in cells. For example, an SPPL3 modulation agent can be an activator, an inhibitor, an agonist, or an antagonist. As another example, the SPPL3 modulation can be the result of gene editing.

Immunotherapy Compositions Targeting CD19 with Modified Glycolysation Profiles

In some aspects, the treatment may include a CD19 CAR T cell therapy that includes a targeting moiety or antibody that recognizes one glycosylation profile of a B cell population associated with one refractive or recurring B cell malignancy. Non-limiting examples of glycosylation profiles of B cell populations associated with refractive or recurring B cell malignancies include hyperglycosylated or hypoglycosylated profiles relative to a wild-type B cell glycosylation profile. In other additional aspects, the treatment may include a CD19-targeted CAR T cell therapy that includes a targeting antibody or moiety configured to recognize at least several glycosylation profiles of CD19 associated with immunotherapy-resistant disease cells, wherein the epitope recognition is tolerant of at least some variation in glycosylation profile.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| | | Bases | Complementary |
|---|---|---|---|
| Base | Name | Represented | Base |
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of a significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein-encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5 (9), 680-688; Sanger et al. (1991) Gene 97 (1), 119-123; Ghadessy et al. (2001) Proc Natl Acad Sci USA 98 (8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6 (log₁₀[Na⁺])+0.41 (fraction G/C content)−0.63 (% formamide)−(600/1). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged | |
| (Acidic): | D E |

Conservative Substitutions III

| | Exemplary |
|---|---|
| Original Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| | Leu, Val, Met, Ala, |
| Ile (I) | Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14 (12): 807-15, describing targeting deoxyribonucleotide sequences; Lee et al. (2006) Curr Opin Chem Biol. 10, 1-8, describing aptamers; Reynolds et al. (2004) Nature Biotechnology 22 (3), 326-330, describing RNAi; Pushparaj and Melendez (2006) Clinical and Experimental Pharmacology and Physiology 33 (5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tₘ of specific internal domains of the siRNA, siRNA length, position of the target sequence within the CDS (coding region), and nucleotide content of the 3′ overhangs.

### Genome Editing

As described herein, CD19 glycosylation or SPPL3 activity or expression can be modulated (e.g., reduced, eliminated, or enhanced) using genome editing. Processes for genome editing are well known; see e.g. Aldi 2018 Nature Communications 9 (1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of CD19 glycosylation or SPPL3 activity or expression by genome editing can result in protection from cancers, particularly immunotherapy-resistant B cell malignancies.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N) 20NGG target DNA sequence). This results in a double-strand break three nucleotides upstream of the NGG motif. The double-strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for cancer to target cells by the removal or addition of CD19 glycosylation or SPPL3 signals (e.g., activate (e.g., CRISPRa), upregulate, overexpress, downregulate CD19 glycosylation or SPPL3 activity or expression).

For example, the methods as described herein can comprise a method for altering a target polynucleotide sequence in a cell comprising contacting the polynucleotide sequence with a clustered regularly interspaced short palindromic repeats-associated (Cas) protein.

### Gene Therapy and Genome Editing

Gene therapies can include inserting a functional gene with a viral vector. Gene therapies for cancer, particularly B cell malignancies, are rapidly advancing.

There has recently been an improved landscape for gene therapies. For example, in the first quarter of 2019, there were 372 ongoing gene therapy clinical trials (Alliance for Regenerative Medicine, May 9, 2019).

Any vector known in the art can be used. For example, the vector can be a viral vector selected from retrovirus, lentivirus, herpes, adenovirus, adeno-associated virus (AAV), rabies, Ebola, lentivirus, or hybrids thereof.

Gene therapy strategies.

| | |
|---|---|
| | Associated experimental |
| Strategy | models |

Viral Vectors

| | | |
|---|---|---|
| Retroviruses | Retroviruses are RNA viruses | Murine model of MPS VII |
| | transcribing their single-stranded | Canine model of MPS VII |
| | genome into a double-stranded | |
| | DNA copy, which can integrate | |
| | into a host chromosome | |
| Adenoviruses (Ad) | Ad can transfect a variety of | Murine model of Pompe, Fabry, |
| | quiescent and proliferating | Walman diseases, |
| | cell types from various species | aspartylglucosaminuria |
| | and can mediate | and MPS VII |
| | robust gene expression | |
| Adeno-associated | Recombinant AAV vectors | Murine models of Pompe, Fabry |
| Viruses (AAV) | contain no viral DNA and can | diseases, |
| | carry ~4.7 kb of foreign | Aspartylglucosaminuria, Krabbe |
| | transgenic material. They | disease, Metachromatic |
| | are replication defective and can | leukodystrophy, MPS I, MPSII, |
| | replicate only while | MPSIIIA, MPSIIIB, MPSIV, |
| | coinfecting with a helper virus | MPSVI, MPS VII, CLN1, CLN2, |
| | | CLN3, CLN5, CLN6 |

Non-viral vectors

| | | |
|---|---|---|
| plasmid DNA | pDNA has many desired | Mouse model of Fabry disease |
| (pDNA) | characteristics as a gene | |
| | therapy vector; there are no | |
| | limits on the size or genetic | |
| | constitution of DNA, it is | |
| | relatively inexpensive to supply, | |
| | and unlike viruses, antibodies | |
| | are not generated | |
| | against DNA in normal | |
| | individuals | |
| RNAi | RNAi is a powerful tool for gene | Transgenic mouse strain |
| | specific silencing that | Mouse models of acute liver |
| | could be useful as an enzyme | failure |
| | reduction therapy or | Mice with hepatitis B virus |
| | means to promote read-through | Fabry mouse |
| | of a premature stop | |
| | codon | |

Gene therapy can allow for the constant delivery of the enzyme directly to target organs and eliminates the need for weekly infusions. Also, the correction of a few cells could lead to the enzyme being secreted into the circulation and taken up by their neighboring cells (cross-correction), resulting in the widespread correction of the biochemical defects. As such, the number of cells that must be modified with a gene transfer vector is relatively low.

Genetic modification can be performed either ex vivo or in vivo. The ex vivo strategy is based on the modification of cells in culture and transplantation of the modified cell into a patient. Cells that are most commonly considered therapeutic targets for monogenic diseases are stem cells. Advances in the collection and isolation of these cells from a variety of sources have promoted autologous gene therapy as a viable option.

The use of endonucleases for targeted genome editing can solve the limitations presented by the usual gene therapy protocols. These enzymes are custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21 st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic agents, or absorption delaying agents. The use of such media and agents for pharmaceutically active substances is well known in the art (see generally Remington's Pharmaceutical Sciences (A. R. Gennaro, Ed.), 21 st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of the agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing cancer, particularly a B cell malignancy or immunotherapy-resistant B cell malignancy, in a subject in need of administration of a therapeutically effective amount of therapeutic agent capable of modulating CD19 glycosylation or SPPL3 expression or activity, so as to improve T cell recognition of CD19, increase activation of CAR T cell effector function, or enhance CAR T anti-tumor cytotoxicity.

Methods described herein are generally performed on a subject in need thereof. A subject in need of the therapeutic methods described herein can be a subject having, diagnosed with, suspected of having, or at risk for developing cancer, particularly a B cell malignancy or immunotherapy-resistant B cell malignancy. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a therapeutic agent capable of modulating CD19 glycosylation or SPPL3 expression or activity is, for example, an amount that would cause the desired therapeutic effect in a subject while minimizing undesired side effects. In various embodiments, an effective amount of the therapeutic agent capable of modulating CD19 glycosylation or SPPL3 expression or activity described herein can substantially inhibit immunotherapy resistance, slow the progress of immunotherapy resistance, or limit the development of immunotherapy resistance.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of the therapeutic agent capable of modulating CD19 glycosylation or SPPL3 expression or activity can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to improve T cell recognition of CD19, increase activation of CAR T cell effector function, or enhance CAR T anti-tumor cytotoxicity.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single-dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from the compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of the therapeutic agent capable of modulating CD19 glycosylation or SPPL3 expression or activity can occur as a single event or over a time course of treatment. For example, the therapeutic agent capable of modulating CD19 glycosylation or SPPL3 expression or activity can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for cancer, particularly B cell malignancies or immunotherapy-resistant B cell malignancies.

A therapeutic agent capable of modulating CD19 glycosylation or SPPL3 expression or activity can be administered simultaneously or sequentially with another agent, such as an antibiotic, an anti-inflammatory, or another agent. For example, a therapeutic agent capable of modulating CD19 glycosylation or SPPL3 expression or activity can be administered simultaneously with another agent, such as an antibiotic or an anti-inflammatory. Simultaneous administration can occur through administration of separate compositions, each containing one or more of a therapeutic agent capable of modulating CD19 glycosylation or SPPL3 expression or activity, an antibiotic, an anti-inflammatory, or another agent. Simultaneous administration can occur through administration of one composition containing two or more of a therapeutic agent capable of modulating CD19 glycosylation or SPPL3 expression or activity, an antibiotic, an anti-inflammatory, or another agent. A therapeutic agent capable of modulating CD19 glycosylation or SPPL3 expression or activity can be administered sequentially with an antibiotic, an anti-inflammatory, or another agent. For example, a therapeutic agent capable of modulating CD19 glycosylation or SPPL3 expression or activity can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22 (3): 659-661, 2008, which is incorporated herein by reference):

HED(mg/kg)=Animal dose(mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m2) is 37, whereas a 20 kg child (BSA 0.8 m2) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the therapeutic agent capable of modulating CD19 glycosylation or SPPL3 expression or activity may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, the therapeutic agent capable of modulating CD19 glycosylation or SPPL3 expression or activity such as a compound described herein may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation have gained significant interest from researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig-derived cells can be transplanted into humans. Xenogeneic cell therapies can involve human cell transplantation into experimental animal models for assessment of efficacy and safety or enable xenogeneic strategies for humans as well.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Screening

Also provided are screening methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about-2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to CD19 glycosylation modulation agents and SPPL3 modulation agents. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Antigen Glycosylation is a Critical Regulator of Car T Cell Efficacy

To characterize how alterations in expression of SPPL3 and post-translational modifications of CD19 lead to antigen-mediated tumor escape from CAR T cell therapy, the following experiments were conducted.

### Abstract

While chimeric antigen receptor (CAR) T cells targeting CD19 can cure a subset of patients with B cell malignancies, most patients treated will not achieve durable remission. Identification of the mechanisms leading to failure is essential to broadening the efficacy of this promising platform. Several studies have demonstrated that disruption of CD19 genes and transcripts can lead to disease relapse after the initial response. However, few other tumor-intrinsic drivers of CAR T cell failure have been reported. Demonstrated herein is that expression of the Golgi-resident intramembrane protease Signal peptide peptidase-like 3 (SPPL3) in malignant B cells is a potent regulator of resistance to CAR therapy. Loss of SPPL3 resulted in hyperglycosylation of CD19 in leukemia and lymphoma, which impaired recognition of CD19, inhibited activation of CAR T cell effector function, and suppressed anti-tumor cytotoxicity. Alternatively, over-expression of SPPL3 caused hypoglycosylation of CD19, also impairing CD19 recognition and enabling resistance. These findings identify that post-translational modifications of full-length CD19 can lead to antigen-mediated tumor escape from CAR T cell therapy.

### Introduction

T cells engineered with chimeric antigen receptors (CARs) targeting the transmembrane protein CD19 have varied success in the treatment of B cell cancers. Response rates in pediatric patients with acute lymphoblastic leukemia (ALL) are high, with >85% achieving complete remission within one month of treatment. Unfortunately, many patients with ALL who achieve remission ultimately relapse. Outcomes for patients with non-Hodgkin lymphoma or chronic lymphocytic leukemia (CLL) are more modest, with overall response rates of 30-50%. These clinical data demonstrate that while curative for some, most patients treated with CAR T cells will not achieve long-term remission.

Failure of CAR T cells can derive from tumor-intrinsic mechanisms, T cell-intrinsic mechanisms, or a combination of both. Several studies have identified T cell-intrinsic features that correlate with therapeutic failure, primarily related to memory differentiation status or expression of exhaustion-associated genes. It was previously reported that defects in cancer cell apoptotic signaling enable resistance to CAR T cell cytotoxicity that then drives the development of T cell dysfunction, implicating both cell types in disease progression. Several other studies have identified mechanisms by which modulation of CD19 surface expression can lead to resistance and relapse. This process, broadly referred to as antigen escape, manifests as an apparent loss of surface CD19 expression by leukemic cells, making CAR T cells “blind” to their presence and permitting disease outgrowth. Antigen escape can occur at the genetic level via the loss of entire CD19 alleles, resulting in failed protein expression, or partial loss of essential genomic regions, resulting in the expression of a truncated protein without the CAR binding epitope. Alternative splicing of transcribed CD19 messenger RNA can result in the elimination of domains necessary for membrane integration or loss of the CAR binding epitope, again resulting in loss of surface expression or loss of regions needed for CAR: antigen engagement. To date, alterations of full-length CD19 protein that lead to resistance have not been reported.

Following translation, proteins can undergo several modifications. Among the most common post-translational modifications of transmembrane proteins is the addition of glycan residues as proteins pass through the endoplasmic reticulum (E.R.) and Golgi apparatus en route to the cell surface. Glycosylation is an iterative process that is regulated by glycan-modifying enzymes found in the E.R. and Golgi and serves an essential role in protein folding and stability. Signal peptide peptidase-like 3 (SPPL3) is an intramembrane aspartyl protease located in the Golgi that cleaves, among other targets, enzymes responsible for adding glycan residues to transiting transmembrane proteins. This cleavage results in the release of the catalytically-active ectodomain of these glycosyltransferases, inhibiting them from adding glycans to proteins passing through the Golgi. Thus, SPPL3 functionally serves to limit protein glycosylation. Antibodies and their derivative single chain variable fragments (scFvs) that comprise CAR antigen-binding domains are variably sensitive to protein glycosylation for epitope recognition, and thus alteration of target antigen glycosylation presents a potential mechanism of escape from CAR T cell recognition.

A genome-wide, CRISPR/Cas9-based loss-of-function screen in the human ALL cell line Nalm6 was performed to identify genes whose function may promote resistance to CD19-targeted CAR T cell cytotoxicity. It was found that disruption of SPPL3 caused CD19 hyperglycosylation which impaired binding of the anti-CD19 scFv, impaired T cell activation, and inhibited cytotoxicity. Over-expression of SPPL3 resulted in CD19 hypoglycosylation which also disrupted anti-CD19 scFv binding and enabled disease resistance. The findings highlight the relevance of protein glycosylation in antigen: receptor interactions and identify post-translational modifications as regulators of CD19-targeted CAR T cell efficacy.

### Methods

### Genome-Wide Knockout Screen

The Brunello sgRNA knockout plasmid library was designed and produced as previously described and Nalm6 cells were engineered as previously described. For screening studies, 2×10⁸ Brunello-edited Nalm6 cells were combined with 5×10⁷ CART19 cells or control T cells (effector: target ratio 1:4) and co-cultured in standard culture media; 5×10⁷ control Nalm6 cells were frozen for genomic DNA analysis. After 24 hours, cultures were collected, underwent dead cell removal, and were prepared for genomic DNA extraction.

### Genomic DNA Extraction and Guide Sequencing

From each screening culture, 3×10⁷-5×10⁷ were flash-frozen as dry cell pellets. At the time of DNA extraction, 6 mL of NK Lysis Buffer (50 mM Tris, 50 mM EDTA, 1% SDS, pH 8) and 30 μL of 20 mg/mL Proteinase K were added to the frozen cell sample and incubated at 55° C. overnight. The following day, 30 μL of 10 mg/mL RNase A, diluted in NK Lysis Buffer to 10 mg/mL and then stored at 4° C., was added to the lysed sample, which was then inverted 25 times and incubated at 37° C. for 30 min. Samples were cooled on ice before the addition of 2 mL of pre-chilled 7.5M ammonium acetate to precipitate proteins. The samples were vortexed at high speed for 20 s and then centrifuged at ≥4,000×g for 10 min. After the spin, a tight pellet was visible in each tube and the supernatant was carefully decanted into a new 15 ml tube. 6 mL 100% isopropanol was added to the tube, inverted 50 times, and centrifuged at ≥4,000×g for 10 min. Genomic DNA (gDNA) was visible as a small white pellet in each tube. The supernatant was discarded, 6 mL of freshly prepared 70% ethanol was added to the tube and inverted 10 times, and centrifuged at ≥4,000×g for 1 min. The supernatant was discarded by pouring, the tube was briefly spun, and the remaining ethanol was removed using a pipette. After air drying for 10-30 min, 500 μL of 1× TE buffer was added, and the tube was incubated at 65° C. for 1 h and at room temperature overnight to fully resuspend the DNA. The next day, the gDNA samples were vortexed briefly, and gDNA concentration was measured. To measure the distribution of sgRNA within each screen arm, Illumina Next Generation Sequencing applied to an amplicon generated from a single targeted PCR of the integrated sgRNA cassette was used. Briefly, all collected gDNA (1000× coverage) was divided into 100 μL PCR reactions with 5 μg of DNA per reaction. Takara ExTaq DNA Polymerase and the default mix protocol with the following PCR program were used: (95° 2 min, (98° 10 sec, 60° 30 sec, 72° 30 sec)×24, 72° 5 min). PCR products were gel purified using the QiaQuick gel extraction kit (Qiagen). The purified, pooled library was then sequenced on a HiSeq4000 with ˜5% PhiX added to the sequencing lane. Quality assessment was done by qubit (for concentration), bioAnalyzer (for size distribution), and Kapa Library Quantification (for clusterable molarity).

### Genome-Wide Screening Data Analysis

To count the number of reads associated with each sgRNA in each Fastq file, the sgRNA targeting sequencing was first extracted using a regular expression containing the three nucleotides flanking each side of the sgRNA 20 bp target. sgRNA spacer sequences were then aligned to a pre-indexed Brunello library (Addgene) using the short-read aligner ‘bowtie’ using parameters (−v 0 −m 1). Data analysis was performed using custom R scripts. Using the guide sequencing data, enrichment and depletion of guide RNAs were analyzed with the Model-based Analysis of Genome-wide CRISPR/Cas9 Knockout (MAGeCK) algorithm using the maximum likelihood estimation (MLE) module.

### CRISPR/Cas9-Guide Design, Genomic Engineering, and Indel Detection

SPPL3 sgRNAs were designed using Benchling (http://Benchling.com). Six guide RNAs targeting early exons were screened for knockout efficiency. Nalm6 and OCI-Ly10 cells were electroporated using the Lonza 4D-Nucleofector Core/X Unit using the SF Cell Line 4-D Nucleofector Kit (Lonza). For Cas9 and sgRNA delivery, the ribonucleoprotein (RNP) complex was first formed by combining 10 μg of Cas9 Protein (Invitrogen) with 5 μg of sgRNA. Cells were spun down at 300×g for 10 min and resuspended at a concentration of 3-5×10⁶ cells/100 μL in the specified buffer. The RNP complex, 100 μL of resuspended cells, and 4 μL of 100 μM IDT Electroporation Enhancer (IDT) were combined and electroporated. After electroporation, cells were cultured at 37° C. for the duration of experimental procedures. Genomic DNA from electroporated cells was isolated (Qiagen DNeasy Blood & Tissue Kit) and 200-300 ng were PCR amplified using Accuprime Pfx SuperMix or Q5 Mastermix (New England Biolabs) and 10 μM forward/reverse primers flanking the region of interest. Primers were designed such that the amplicon was at a target size of about 1 kb. PCR products were gel purified and sequenced, and trace files were analyzed to determine KO efficiency. R2 values were calculated, reflecting the goodness of fit after non-negative linear modeling by TIDE software.

### General Cell Culture

Unless otherwise specified, cells were grown and cultured at a concentration of 1×10⁶ cells/mL of standard culture media (RPMI 1640+10% FCS, 1% penicillin/streptomycin, 1% HEPES, 1% non-essential amino acids) at 37° C. in 5% ambient CO₂.

### Lentiviral Vector Production and Transduction of Human Cells

Replication-defective, third-generation lentiviral vectors were produced using HEK293T cells (ATCC ACS-4500). Approximately 10×10⁶ cells were plated in T175 culture vessels in DMEM+10% FCS culture media and incubated overnight at 37° C. 18-24 h later, cells were transfected using a combination of Lipofectamine 2000 (96 μL, Invitrogen), pMDG.1 (7 μg), pRSV.rev (18 μg), pMDLg/p.RRE (18 μg) packaging plasmids and 15 μg of expression plasmid. Lipofectamine and plasmid DNA were diluted in 4 mL Opti-MEM media prior to transfer into lentiviral production flasks. At both 24 and 48 h following transfection, culture media was isolated and concentrated using high-speed ultracentrifugation (8,500×g overnight). For T cell engineering, CD4 and CD8 T cells were isolated from Miltenyi PBMC packs and combined at a 1:1 ratio, and activated using CD3/CD28 stimulatory beads (Thermo-Fisher) at a ratio of 3 beads/cell and incubated at 37° C. overnight. The following day, CAR lentiviral vectors were added to stimulatory cultures at an MOI of 3. Beads were removed on day 6 of stimulation, and cells were counted daily until growth kinetics and cell size demonstrated they had rested from stimulation. For cancer cell engineering, vectors were combined with cells at an MOI of 2.

### Co-Culture Assays

For cytotoxicity assays, CAR T cells were combined with target cells at various E:T ratios, and co-cultures were evaluated for an absolute count of target cells by flow cytometry. All co-cultures were established in technical triplicate. Cultures were maintained at a concentration of 1e6 total cells/mL. For re-exposure assays, CAR+ T cells were sorted by fluorescence-assisted cell sorting using a truncated CD34 selection marker encoded in the CAR plasmid backbone. T cells were then recombined with target leukemia cells at an effector: target ratio of 1:4 and killing was measured as described. For activation marker studies, CAR or Jurkat T cells and Nalm6 cells were combined at an E:T ratio of 1:4 and evaluated by flow cytometry the following day. Jurkat cells were engineered to express a dual fluorescence reporter system indicating activation of transcription factor activity as previously described. For degranulation assays (CD107a assessment), T cells were combined with Nalm6 as described and combined with an antibody cocktail of CD107a-PECy7 (clone H4A3, Biolegend) and stimulatory antibodies against CD28 (eBiosciences) for one hour. Intracellular protein transport was halted by the addition of GolgiStop (BD Biosciences) and cells were incubated for an additional three hours. Cells were then harvested and stained for CD34 (BD #555824) and analyzed by flow cytometry.

### Flow Cytometry

Cells were resuspended in FACS staining buffer (PBS+3% fetal bovine serum) using the following antibodies: CD3 (clone OKT3, BD Biosciences), PD-1 (clone EH12.2H7, BioLegend), Tim3 (clone 7D3, BD Biosciences), CD22 (clone HIB22, BD Biosciences), CD19 (clone FMC63, Novus Biologicals; clone HIB19, BD Biosciences). CARs transduction was evaluated by staining for a truncated CD34 selection marker located downstream of a P2A ribosomal skip sequence from the CAR transgene. Data were acquired on an Attune NxT cytometer (Thermo). All data analysis was performed using FlowJo 9.0 software (FlowJo, LLC).

### Western Blotting

Nalm6 and OCI-Ly10 cells were counted and 5×10⁶ cells were washed in cold PBS. Cell pellets were resuspended in RIPA lysis buffer supplemented with phosphatase and protease inhibitors and incubated on ice for 15 minutes, followed by centrifugation at 14000×g for 15 minutes. Lysate concentration was quantified using the Pierce BSA Protein Assay Kit (Thermo), combined with 4× LDS buffer, denatured at 100° C. for 10 minutes, and then reduced to a final concentration of 20% beta-mercaptoethanol. 10-20 μg of protein was loaded into each well of a Bis-Tris gel (either 4-12% gradient, 6% or 15%) and proteins were separated using standard electrophoresis followed by transfer to nitrocellulose membranes. Proteins were labeled with SPPL3 (EMD Millipore), CD19, CD22, Actin, or GAPDH (all from CellSignaling), followed by secondary antibody staining and visualization.

### Protein Modeling

Prediction of CD19 structure was performed using the Phyre2 web portal for protein modeling, prediction, and analysis. The resulting predicted CD19 structure was further analyzed and visualized using UCSF Chimera. Sequences used for predictive modeling and analysis were derived from the Protein Database entry 6AL5.

### Statistical Analysis

All data presented are representative of independent experiments using T cells derived from between two to five independent donors, except for the CRISPR knockout screen (performed once with four biological replicates). All cytotoxicity studies and flow-based protein expression studies were performed in technical triplicate. Comparisons between the two groups were performed using either a two-tailed unpaired Student's t-test. Comparisons between more than two groups were performed by two-way analysis of variance (ANOVA) with Bonferroni correction for multiple comparisons. All results are represented as mean±standard error of the mean (s.e.m.).

### Results

### Loss of SPPL3 Results in Resistance to CART19

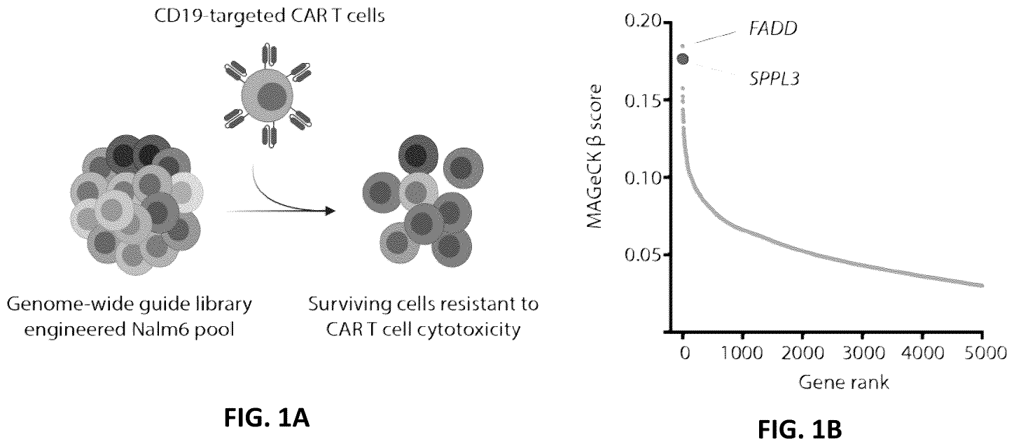

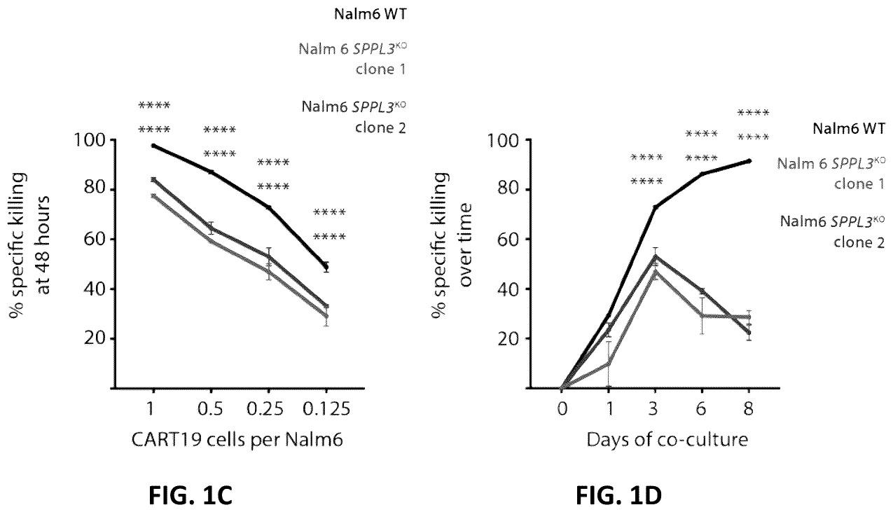

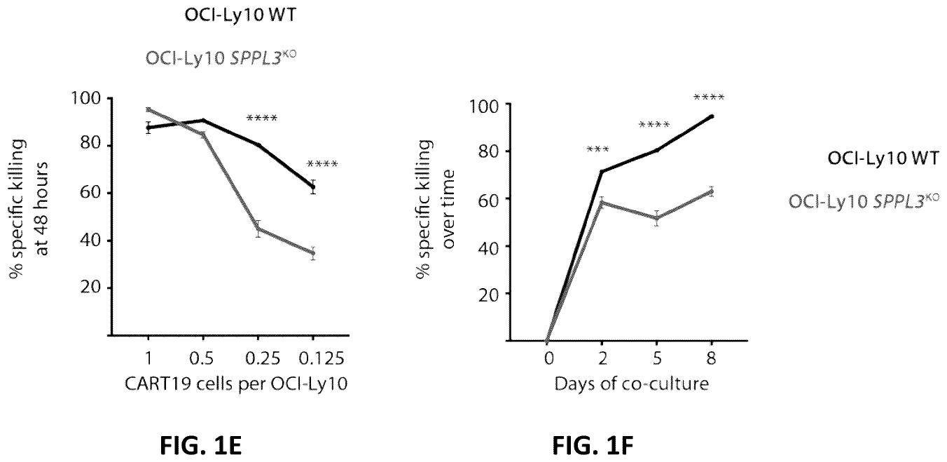

The human B-ALL cell line Nalm6 was engineered with the Brunello genome-wide guide RNA library to enable the loss of function of a single gene within each Nalm6 cell. Following engineering, cells were combined with either control un-engineered T cells or CD19-targeted CAR T cells (CART19). Co-cultures were collected 24 hours later and underwent next-generation sequencing to identify which guide RNAs had been enriched in the surviving CART19-exposed cells, reflecting that loss of gene function promoted resistance to CART19 killing (FIG. 1A). As reported previously, the most enriched gene was FADD, encoding a membrane-proximal protein that initiates the apoptotic cascade upon binding of death receptors to their ligands. The second-most enriched gene was SPPL3 (FIG. 1B), encoding a protease that regulates the glycosylation of secreted and transmembrane proteins. To validate SPPL3's role in resistance to CART19, genomic SPPL3 in Nalm6 was disrupted using de novo designed guide RNAs targeting either exon 2 or 4 (of 11 total exons) and expanded single-cell clones that had biallelic disruption (clone 1 and 2, respectively). It was confirmed that these clones lacked SPPL3 protein expression and that loss of SPPL3 did not enable an intrinsic growth advantage in Nalm6 cells. To directly assess the impact of SPPL3 loss on CART19 cytotoxic efficacy, co-cultures of either SPPL3 wild-type (WT) or SPPL3KO Nalm6 with CART19 were established and Nalm6 survival was monitored over time. These studies confirmed that SPPL3KO Nalm6 were resistant to CAR T cell killing both early during co-culture (48 hours, FIG. 1C) and over time (FIG. 1D). To validate that resistance was not unique to ALL, SPPL3 was disrupted in the CD19+ diffuse large B cell lymphoma line OCI-Ly10. As observed with Nalm6, SPPL3KO OCI-Ly10 were also resistant to CART19 killing (FIGS. 1E and 1F), demonstrating that loss of SPPL3 enabled resistance in two distinct histologies of CD19+ malignancy.

### Loss of SPPL3 in ALL Impairs CART19 Activation

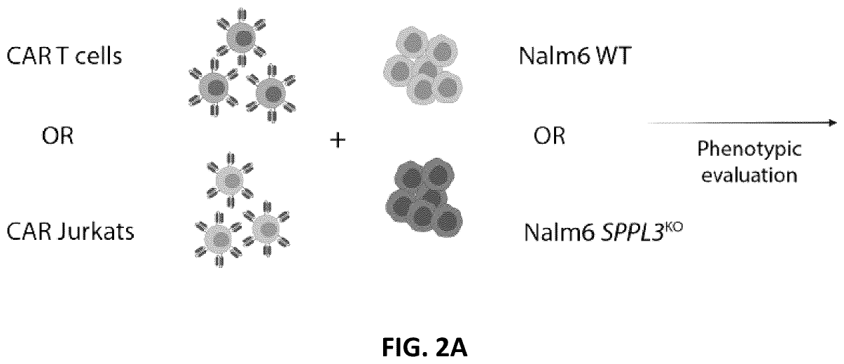

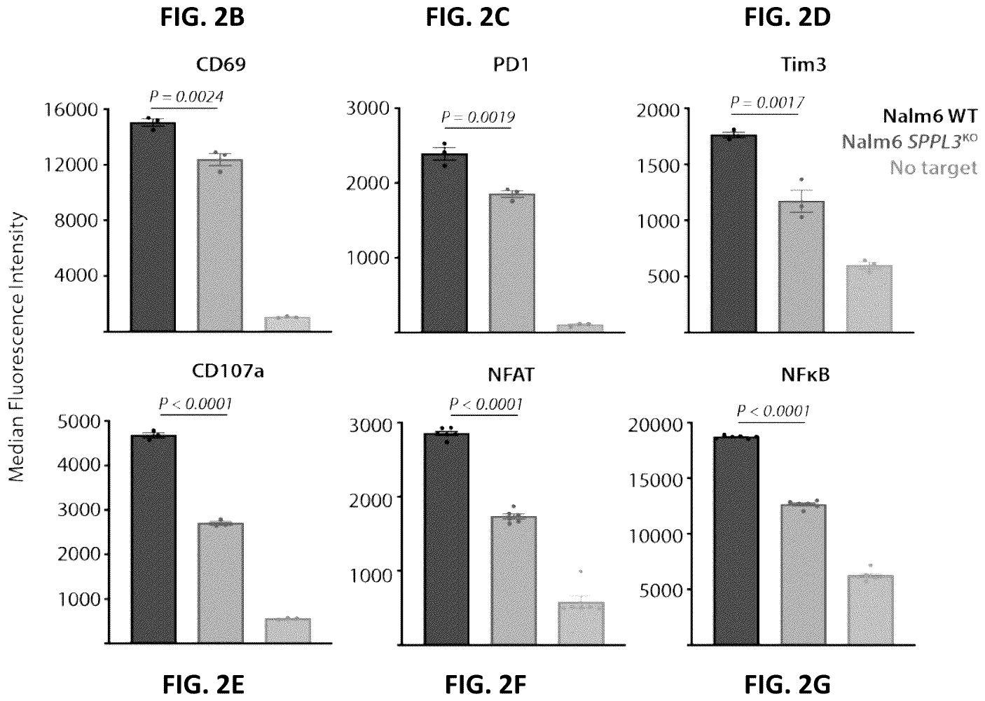

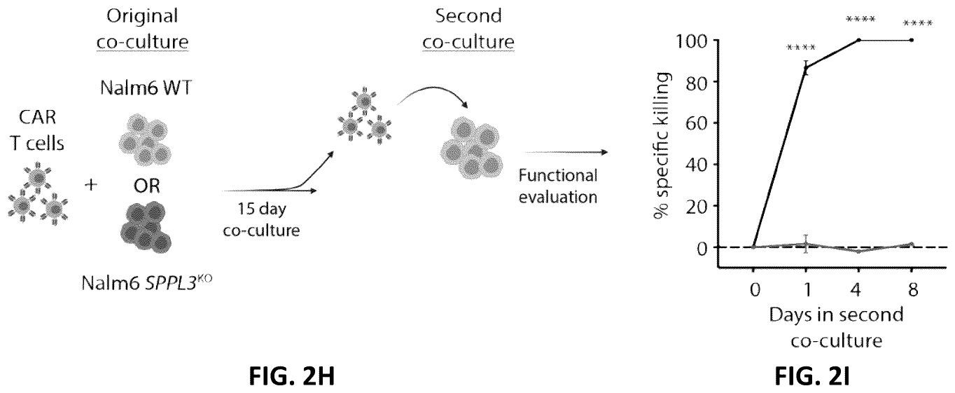

Disruption of SPPL3 in malignant B cells could lead to resistance by enabling failure of the cancer cell death machinery or from an extrinsic impairment of CAR T cell cytotoxic function. It was hypothesized that the resistance observed was unlikely to result from global impairments in cancer cell apoptotic machinery, which would require extensive functional changes in many individual pathways, but instead resulted from an induced impairment of T cell function. To evaluate this, CART19 was combined with either WT or SPPL3KO Nalm6, and the expression of CD69, PD1, and Tim3 were measured; CD69, PD1, and Tim3 are proteins that are expressed on the T cell surface upon receptor-driven activation (FIG. 2A). As compared to cells exposed to WT Nalm6, CART19 exposed to SPPL3KO Nalm6 expressed lower levels of all three activation markers following overnight co-culture (FIGS. 2B, 2C, and 2D). Expression of CD10⁷a (LAMP-1), a surrogate marker of lymphocyte degranulation, was also lower in CART19 exposed to SPPL3KO Nalm6 (FIG. 2E). Next, co-cultures were established by combining WT or SPPL3KO Nalm6 with immortalized T cells (Jurkat) engineered to express the CD19 CAR (FIG. 2A). These Jurkats were previously engineered with a fluorescent reporter system that transcribes GFP or eCFP upon activation of the central T cell transcription factors NFAT or NFκB, respectively. Consistent with the findings in primary T cells, CAR19 Jurkat cells exposed to SPPL3KO Nalm6 demonstrated less transcription factor activation (FIGS. 2F and 2G).

While signifying a quantitative decrease in receptor-driven stimulation, these findings did not demonstrate a complete lack of CAR-driven activation upon exposure to SPPL3KO Nalm6, as indicated by the increase in activation marker expression compared to control, CAR-negative cells (FIGS. 2B, 2C, 2D, 2E, 2F, and 2G). It was previously identified that partial resistance to CAR T cell cytotoxicity enables the survival of some target cells, which leads to the persistence of residual antigens. This persistence drives the development of T cell dysfunction, leading to a two-phased mechanism of therapeutic failure despite only partial resistance to cytotoxicity. To evaluate if this mechanism was responsible for the resistance observed with SPPL3 loss, two parallel cultures of CART19 with either WT or SPPL3KO Nalm6 were established (FIG. 2H). These cultures were maintained for 15 days, at which time no residual WT Nalm6 remained but large quantities of SPPL3KO Nalm6 persisted. CART19 cells were then isolated from these cultures and re-combined with WT Nalm6 to interrogate CART19 cytotoxic function. CART19 cells originally exposed to WT Nalm6 rapidly cleared re-challenge, while CART19 cells originally exposed to SPPL3KO Nalm6 were profoundly dysfunctional, with a complete inability to control WT Nalm6 upon re-challenge (FIG. 2I). Together, these data demonstrate that CART19 cells undergo less antigen-driven activation by SPPL3KO Nalm6 and become progressively dysfunctional as a result of leukemic persistence, suggesting that SPPL3KO Nalm6 resistance may result from a combination of partial evasion of T cell killing followed by T cell failure.

### CD19 Glycosylation Impacts Binding of FMC63

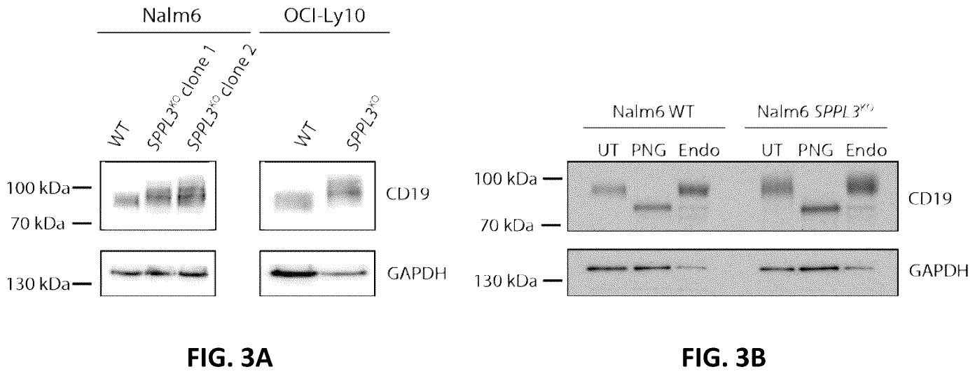

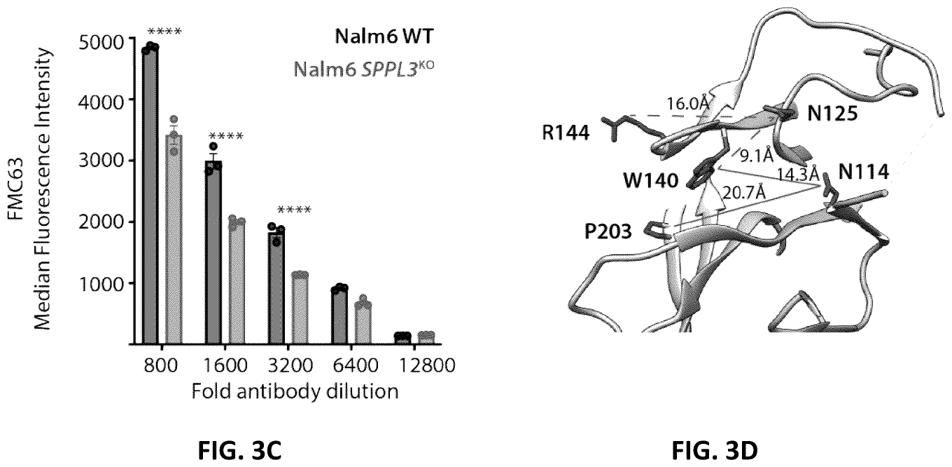

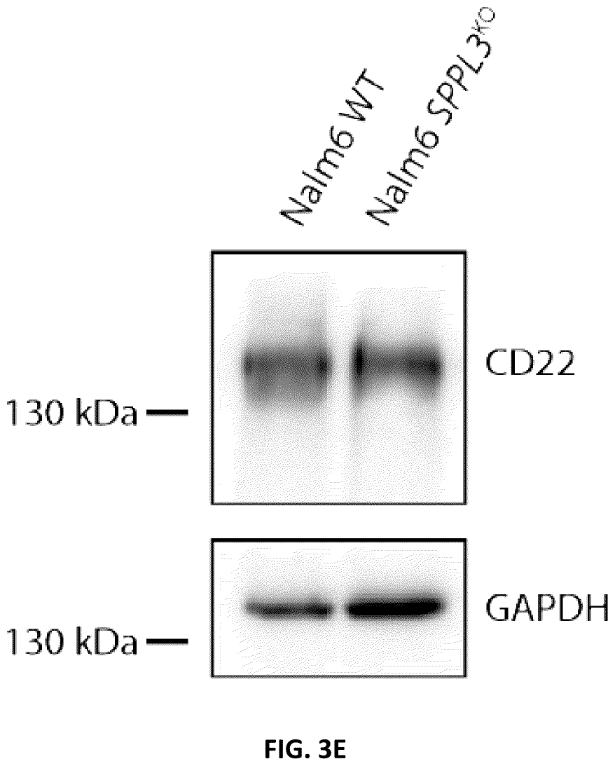

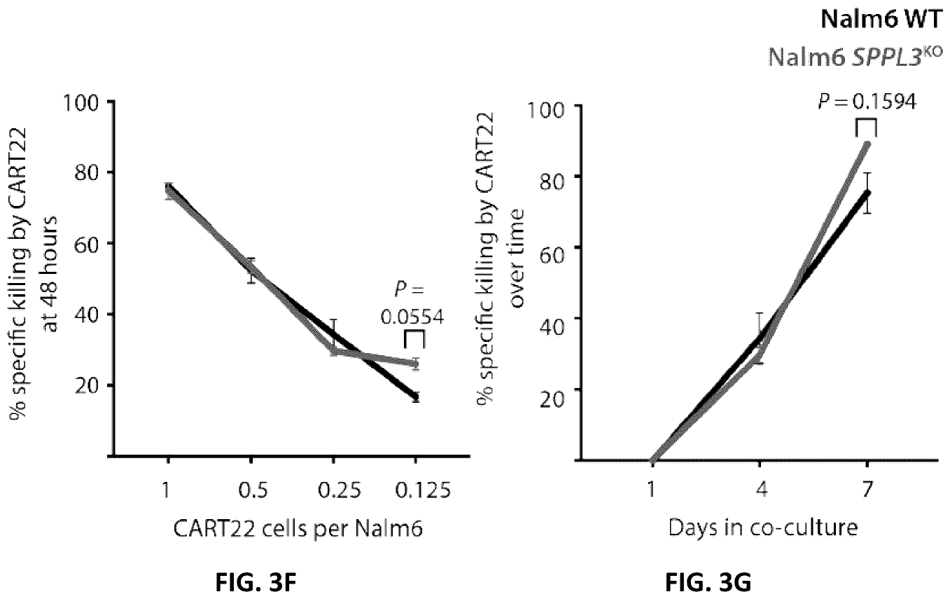

While SPPL3's function has not been comprehensively defined, recent studies identified one of its primary roles as a Golgi-resident intramembrane aspartyl protease is to cleave enzymes that add glycans to extracellular asparagine residues, referred to as N-glycosylation. The extracellular domain of CD19 contains seven asparagines, five of which are glycosylated. Analysis of protein lysates from both Nalm6 and OCI-Ly10 cells revealed that CD19 had a higher molecular weight in the setting of SPPL3 loss (FIG. 3A). To determine if increased glycosylation was responsible for this increase in CD19 size, Nalm6 lysates were treated with two distinct glycosylases, peptide: N glycosidase F (PNG) or endoglycosidase H (Endo). These enzymes have distinct enzymatic activity: PNG cleaves all (high mannose and complex) N-linked glycosyl residues, while Endo only cleaves core (high mannose) residues added in the endoplasmic reticulum. This approach allowed one to simultaneously determine if the increase in molecular weight was a result of N-glycosylation and the complexity of that glycosylation, a reflection of the organellar origin. PNG treatment resulted in a significant reduction in CD19 size in both WT and SPPL3KO, while Endo had minimal impact on CD19 from either cell type (FIG. 3B). These findings indicate that hyperglycosylation, and not another form of post-translational modification, was responsible for the shift in CD19's molecular weight in the setting of SPPL3 loss. Further, these data confirm that CD19 hyperglycosylation results from the addition of complex glycans added in the Golgi and not high mannose residues, consistent with SPPL3 activity.

Given that antibodies and scFvs can be sensitive to glycoprotein structure, it was hypothesized that hyperglycosylation may impair CAR binding of CD19. To evaluate this, Nalm6 cells were stained with the same anti-CD19 antibody used to construct the CAR antigen-binding domain (clone FMC63). Notably, this is the same clone used to construct all FDA-approved CD19-targeted CAR products. At high antibody concentrations (1:100-1:400 dilution) there was no difference in FMC63 binding to CD19 on WT or SPPL3KO Nalm6, however at lower concentrations (1:800-1:3200 dilution) there was a notable reduction in the detection of CD19 on SPPL3KO Nalm6 (FIG. 3C). We also interrogated the ability of a distinct CD19-directed antibody (clone HIB19) to recognize CD19 was also interrogated, and less binding at all antibody concentrations on SPPL3KO Nalm6 was observed, suggesting that FMC63 is not uniquely sensitive to changes in glycosylation of CD19. A previous study identified that CD19 residues W140, R144, and P203 are essential for FMC63 binding. Modeling of the FMC63 binding epitope revealed two proximal asparagine residues, one of which is normally N-glycosylated (N125) and one of which is not (N114, FIG. 3D). Predicted intramolecular distance measurements reveal that N125 is closer to the binding epitope than N114, suggesting that N125 is more likely responsible for the disruption of the FMC63 binding epitope. This is consistent with known Golgi-resident glycosyltransferase functionality-sequential addition of complex glycans to residues that have already undergone some glycosylation in the E.R. to create large extending branches, as opposed to de novo modification of non-glycosylated residues.

While these distinctions in CD19 glycosylation correlated with anti-CD19 antibody binding, T cell activation, and CART19 cytotoxic function, it remained possible that loss of SPPL3 enabled resistance via a distinct mechanism. To confirm that resistance was dependent on glycosylation, a model in which target protein glycosylation was not impacted by SPPL3 loss was sought. Similar to CD19, CD22 is broadly expressed by B-lineage cells and is a well-described CAR target in ALL. In contrast to CD19, the deletion of SPPL3 did not change CD22 electrophoretic mobility, suggesting no change in its glycosylation status. SPPL3KO also did not impact anti-CD22 antibody binding on Nalm6 cells. Consistent with this observation, in vitro co-cultures demonstrated no difference in CD22-directed CAR T cell cytotoxicity against SPPL3KO and WT Nalm6, confirming that loss of SPPL3 did not enable resistance to CART22 (FIGS. 3F and 3G). Together, these findings support the hypothesis that alteration of CD19 glycosylation is responsible for resistance to CART19.

### Hypoglycosylation of CD19 Impairs CART19 Efficacy

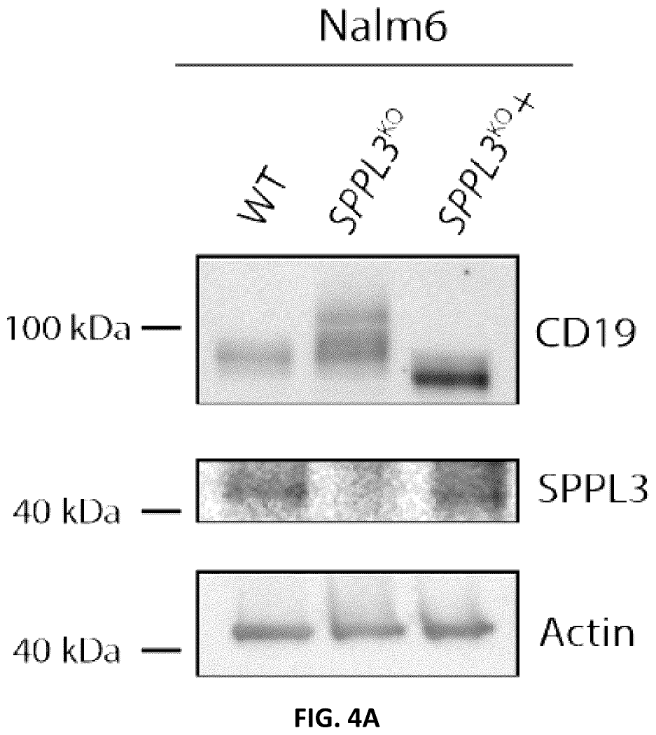

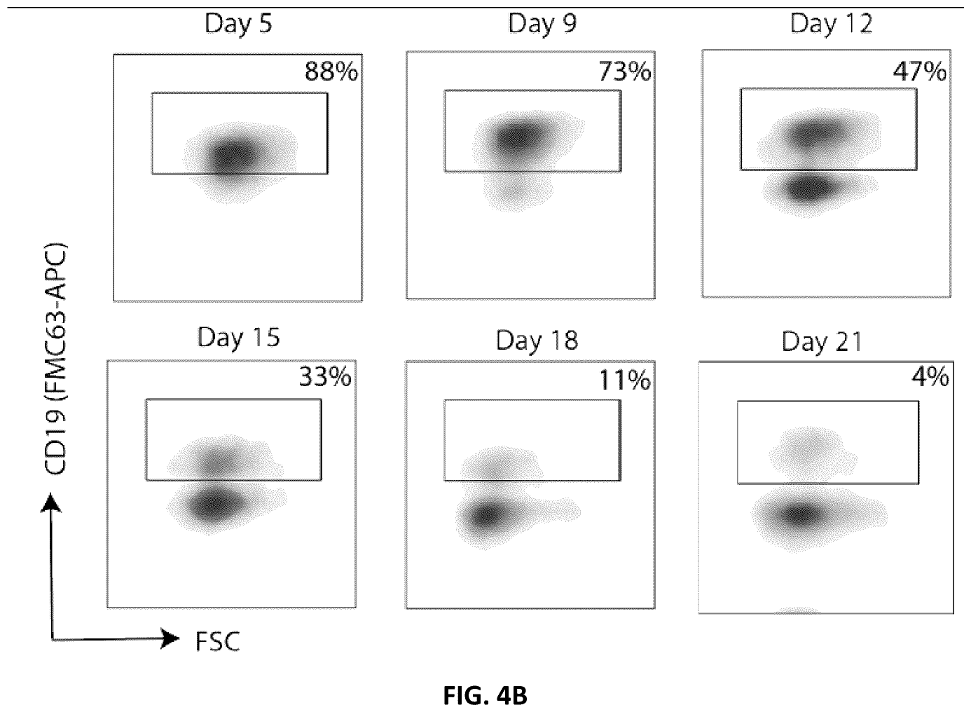

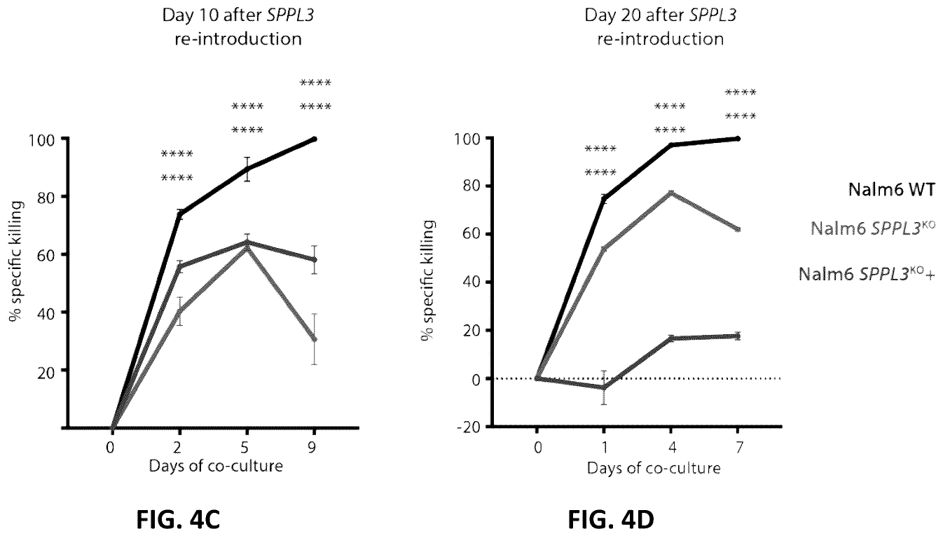

Over-expression of SPPL3 has been shown to cause protein hypoglycosylation via increased glycosyltransferase cleavage, providing a convenient approach to modulate glycosylation on SPPL3 targets. To further validate the role of CD19 glycosylation in the regulation of CART19 efficacy, SPPL3 was over-expressed in SPPL3KO Nalm6 (SPPL3KO+) using a lentiviral expression vector containing a constitutively-active promoter (EF1α) followed by SPPL3 and a CD34 selection marker. CD34+ cells were purified and it was found that over-expression of SPPL3 resulted in the reduction of CD19 size compared to both wild-type and SPPL3KO cells (FIG. 4A), consistent with hypoglycosylation. Evaluation of these cells revealed a progressive impairment in FMC63 binding to CD19 on SPPL3KO+ cells over time after engineering, with the emergence of a clear CD19-negative population that increased in proportion (FIG. 4B). Staining with the HIB19 anti-CD19 antibody clone revealed a similar shift in binding with SPPL3 over-expression. Consistent with this progressive loss of CD19 detection by FMC63, SPPL3KO+Nalm6 became increasingly resistant to CART19. SPPL3KO+Nalm6 combined with CART19 10 days after engineering with the over-expression vector demonstrated resistance that was similar to the resistance observed for SPPL3KO Nalm6 (FIG. 4C). In contrast, co-culture of SPPL3KO+Nalm6 collected 20 days after engineering with CART19 revealed profound resistance, with no observable CART19 cytotoxicity (FIG. 4D). This resistance was maintained even at high effector: target ratios. These findings indicate that in addition to hyperglycosylation, hypoglycosylation of CD19 impairs the binding of FMC63 and enables resistance to CART19.

### Discussion

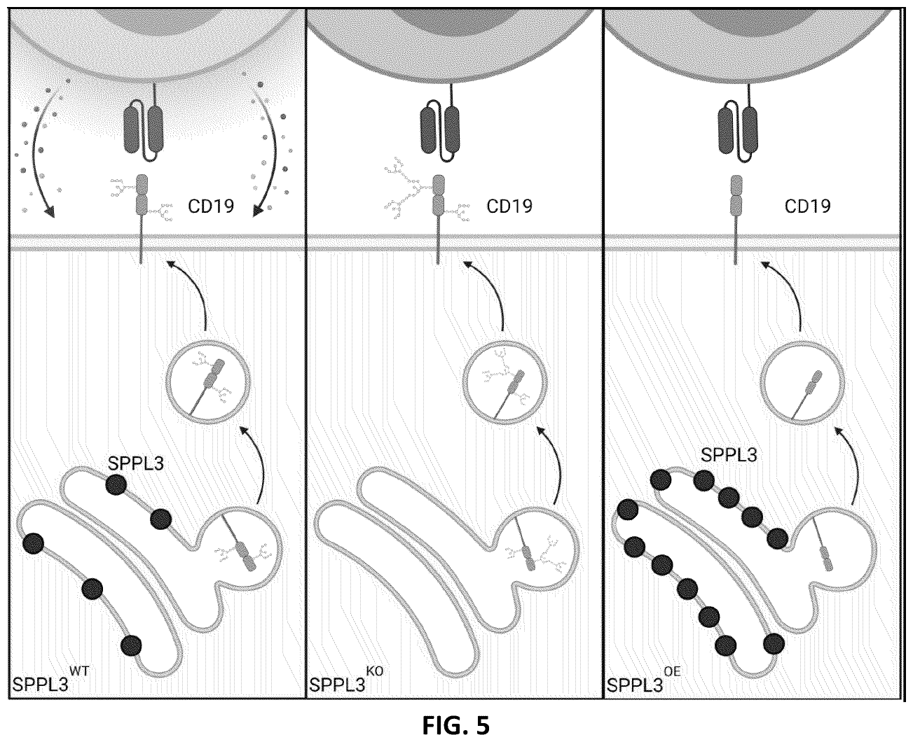

Understanding mechanisms of resistance to CAR T cells is fundamental to improving the efficacy of this platform in both hematologic and solid malignancies. Beyond enhancing therapeutic activity, the identification of tumor-intrinsic features that lead to resistance will allow for more appropriate patient selection, sparing patients with tumors that are unlikely to respond to ineffective therapies. Gene and transcript-level alterations have previously been shown to impair CAR recognition of CD19 leading to antigen escape. Here the mechanisms that lead to antigen escape are expanded to include post-translation modifications of CD19. It was found that modulation of SPPL3, either loss or over-expression, resulted in changes to CD19 glycosylation that were both associated with impairment of anti-CD19 antibody binding and anti-CD19 CAR T cell function (FIG. 5).

A characteristic feature of malignant transformation is a global alteration in protein glycosylation. This has been specifically extended to N-linked glycosylation of transmembrane proteins. As a result of this intrinsic alteration of surface protein glycosylation, some malignant cells may be predisposed to evade CAR binding. Validating this speculation, a novel CD19 isoform that contains an in-frame deletion of Tyr260 expressed in ALL cells from a patient who was resistant to CART19 was described. In this report, it was observed that when transgenically expressed in human cells, CD19 ΔTyr260 was hypoglycosylated, demonstrated impaired binding of CD19 antibodies, and enabled resistance to CART19, mirroring the mechanistic observations in SPPL3 over-expressing cells. These data provide clinical evidence that alterations in CD19 glycosylation can indeed lead to CART19 failure.

The sensitivity of antibodies and single-chain variable fragments to glycosylation patterns on glycoproteins is well-defined. Indeed, broad efforts are focused on the development of CARs that target specific glycan modifications on cell surface proteins. Similar to the conceptual foundation of these studies, these data highlight that modification of protein glycosylation can alter CAR-driven T cell function, in this case limiting CAR activity instead of improving target specificity. Previous in vitro studies have found that deglycosylation of the extracellular CD19 asparagine residues does not impair FMC63 binding. While these studies were performed using purified proteins and not cellular systems, the data suggest that hypoglycosylation may not directly impair anti-CD19 antibody (or CAR) binding but instead may lead to resistance by another mechanism, such as preventing CD19 expression on the cell surface. Further studies exploring the impact of hypoglycosylation on CD19 epitope structure and surface expression are needed to understand these biochemical dynamics. Regardless of the precise mechanism, however, the findings indicate that decreased glycosylation prevents FMC63 interaction with CD19 and results in profound resistance to CART19.

While the studies focus on the manipulation of SPPL3 expression, the data indicate that resistance is not inherent to SPPL3 itself but to its role in regulating CD19 glycosylation. Loss of SPPL3 does not impact the efficacy of CART22, and its over-expression also enables resistance to CART19. As such, the impact of these findings derives from the observation that CD19-targeted CAR T cells are highly-sensitive to target glycosylation status. There are likely several other cellular pathways that can regulate CD19 glycosylation and impact tumor cell sensitivity to CAR therapy. Given its role in cleaving glycosylation regulators, SPPL3 serves a more centralized, but indirect, role in regulating CD19 glycosylation, and thus served as an ideal system to identify this novel mechanism of resistance.

In summary, it was demonstrated that alterations in CD19 glycosylation that result from the modified expression of SPPL3 lead to failed CAR T cell function and disease resistance. These findings identify alteration of post-translational modifications as an additional mechanism of antigen escape for cell-based immunotherapy. Studies to further validate the relationship between target glycosylation and clinical outcomes after CAR T cell therapy are essential and will inform strategies to better identify patients who may derive the most benefit from this therapy.

### Example 2: Mechanisms of Antigen Escape: Discovery of a Novel CD19 Point Mutation that Renders Leukemic Tumor Cells Resistant to CD19 Bispecific T Cell Engager and Anti-CD19 Chimeric Antigen Receptor (Car) T Cell Therapy

To characterize how a mutation affecting CD19 glycosylation leads to blinatumomab and anti-CD19 CAR T cell resistance, the following experiments were conducted.

Described herein is the discovery of a novel point mutation, an in-frame deletion within the extracellular domain of CD19 at Tyr260 (CD19ΔTyr260) in B-ALL that potentially emerged during treatment with blinatumomab, resulting in resistance to blinatumomab and subsequent anti-CD19 CAR T cell therapy. This mutation does not hinder CD19ΔTyr260 expression on the tumor cell surface but alters CD19ΔTyr260 glycosylation dramatically, rendering lymphoblasts resistant to blinatumomab and anti-CD19 CAR T cell killing.

KTE-X19 is an autologous anti-CD19 chimeric antigen receptor (CAR) T-cell therapy under investigation in the ZUMA-3 Phase 1/2 study (NCT02614066) for adult patients with relapsed/refractory (R/R) B-cell acute lymphoblastic leukemia (B-ALL). Blinatumomab is a CD19 bispecific T-cell engager that is approved for the treatment of patients with R/R B-ALL. The emergence of CD19-negative clones involving several different mechanisms is a cause of relapse in 10%-20% of patients receiving blinatumomab or anti-CD19 CAR T for B-ALL. Here, the discovery of the novel point mutation in CD19 at Tyr260 (CD19ΔTyr260) in B-ALL is described that appeared to emerge during treatment with blinatumomab, resulting in resistance to blinatumomab and subsequent anti-CD19 CAR T-cell (KTE-X19) therapy.

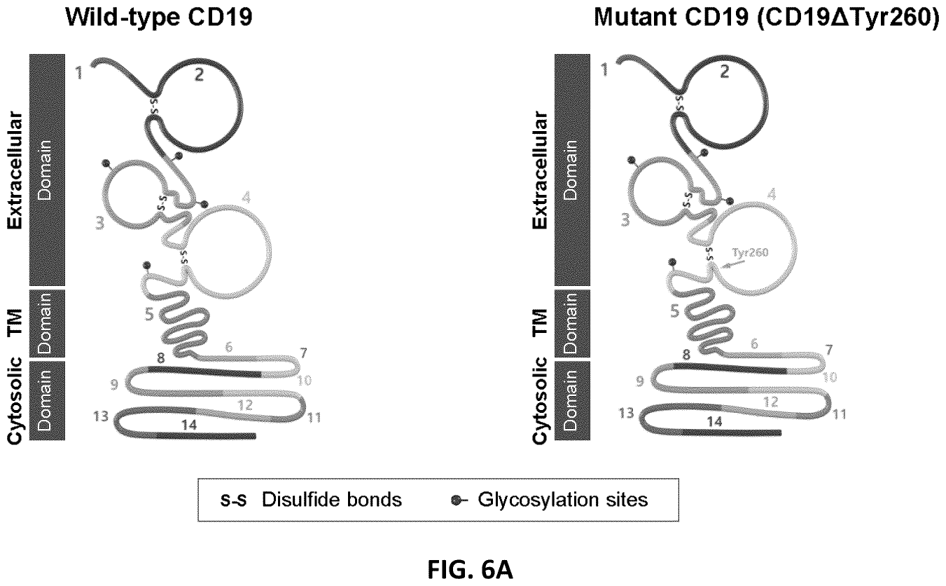

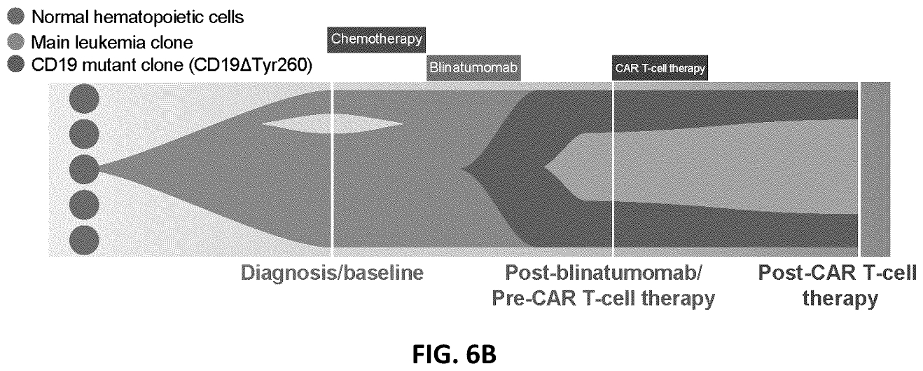

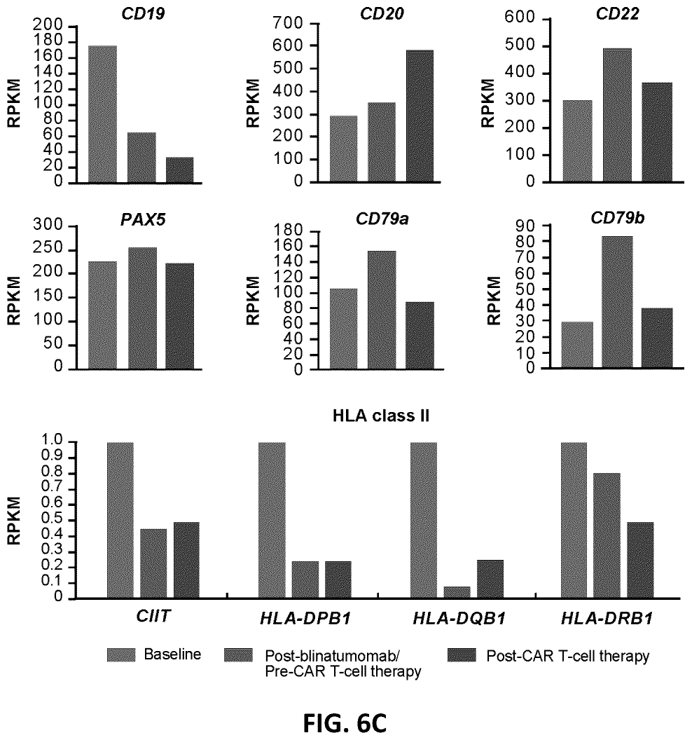

A 60-year-old female with B-ALL was treated with chemotherapy followed by 2 cycles of blinatumomab as initial therapy. She relapsed after the second cycle of blinatumomab. She was then enrolled in ZUMA-3 and received a single infusion of anti-CD19 CAR T cells/kg at the target dose but did not respond to KTE-X19. Although local pathology concluded that pre-KTE-X19 B lymphoblasts were uniformly CD19dim by flow cytometry (clone J3-119), detailed retrospective analysis of biobanked samples from diagnosis and at relapse post-blinatumomab using the murine monoclonal antibody (mAb) FMC63, the same parent antibody used to derive the single-chain variable fragment of KTE-X19, revealed that CD19 was not detectable in post-blinatumomab/pre-KTE-X19 or post-KTE-X19 B lymphoblasts. Using targeted RNA sequencing, an in-frame deletion within the extracellular domain of CD19 at Tyr260 (CD19ΔTyr260) was identified (FIG. 6A).

To understand the clonal evolution of B-ALL and the timing of the emergence of the mutation, enhanced exome and RNA sequencing was performed. Bone marrow samples from diagnosis, post-blinatumomab/pre-KTE-X19, and post-KTE-X19 therapy were analyzed. Clonal inference and sequencing data revealed that deletion of one CD19 allele and mutation in the second CD19 allele potentially emerged during blinatumomab and before KTE-X19 infusion (FIG. 6B). The mutant CD19 allele frequency was 90.47% post-blinatumomab/pre-CAR T-cell therapy and 82.75% post-CAR T-cell therapy. Digital droplet PCR with a sensitivity of 1:100,000 confirmed that the mutation was absent at diagnosis. A second CD19 mutation, resulting in a stop-gain at amino acid 120, was observed in other post-blinatumomab/pre-CAR T-cell and post-CAR T-cell samples from the same patient, but only at low variant allele frequency (3%), suggesting that it did not provide a clonal advantage in the context of treatment.

Because this was a single case, there was limited statistical power to identify differentially expressed genes between the primary tumor and post-therapy time points. Therefore, specific genes or pathways that we hypothesized might be involved in therapy response were examined. CD19 gene expression levels decreased after blinatumomab treatment, whereas CD20, CD22, CD79a, and CD79b expression increased (FIG. 6C). Interestingly, expression of major histocompatibility complex (MHC) class II genes and class II transactivator (CIITA), a transcription factor for MHC class II genes, decreased post-blinatumomab and post-CAR T-cell therapy, suggesting a second mechanism of resistance in this patient (FIG. 6C). Although how downregulation of MHC class II may contribute to resistance to T-cell engagers and CAR T cells is not yet clear, downregulation of MHC class II can potentially suppress antigen presentation by malignant cells preventing activation of non-CAR “bystander” T cells. Patterns of expression in other immune-related genes, T-cell modulatory genes, T-cell ligands, and B-cell receptor signaling pathways were variable.

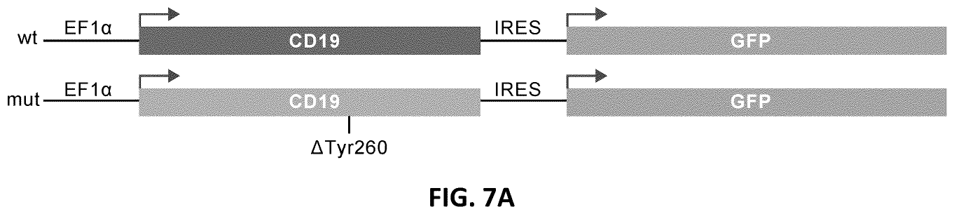

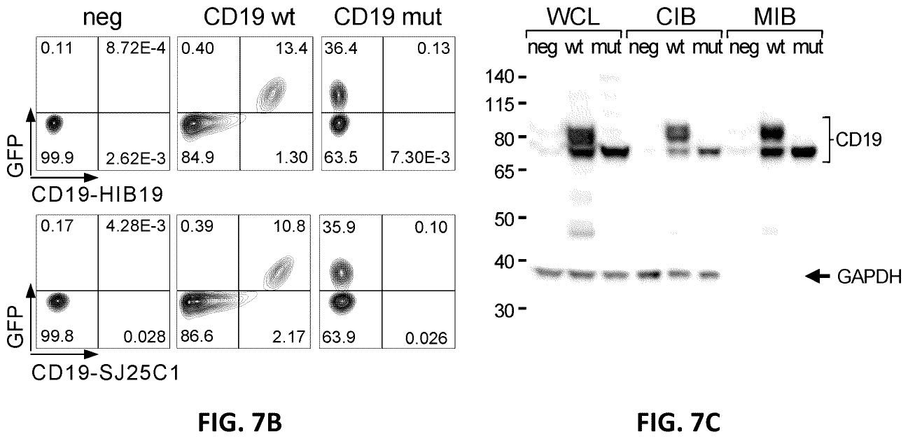

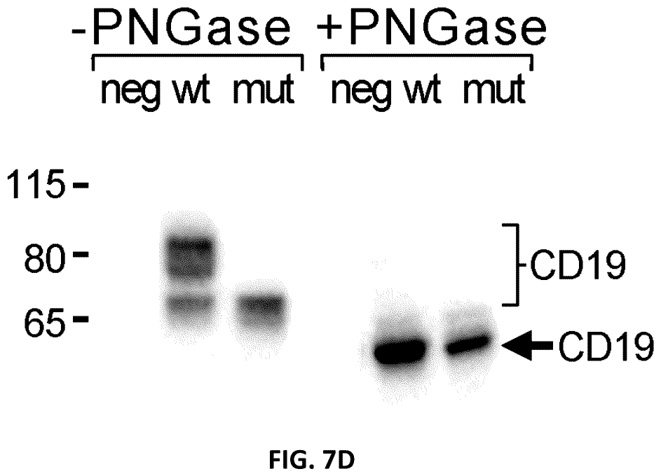

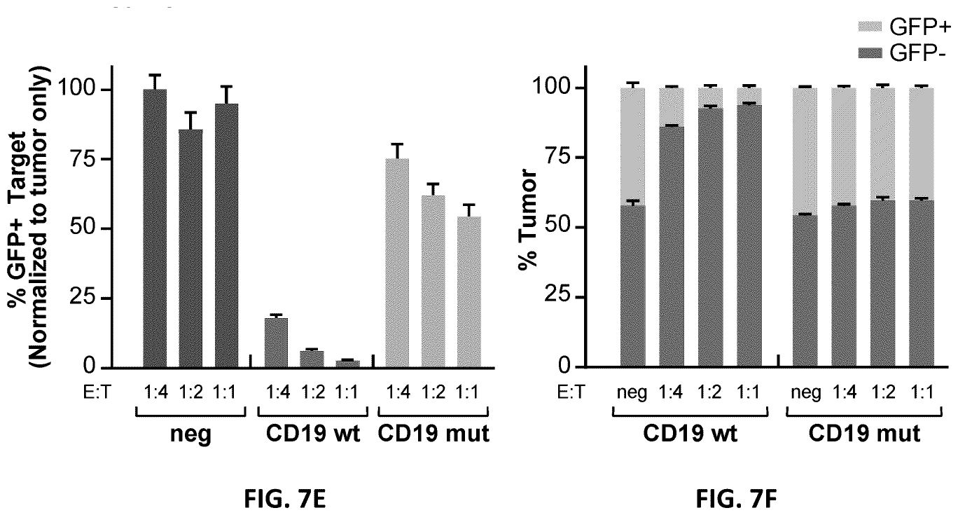

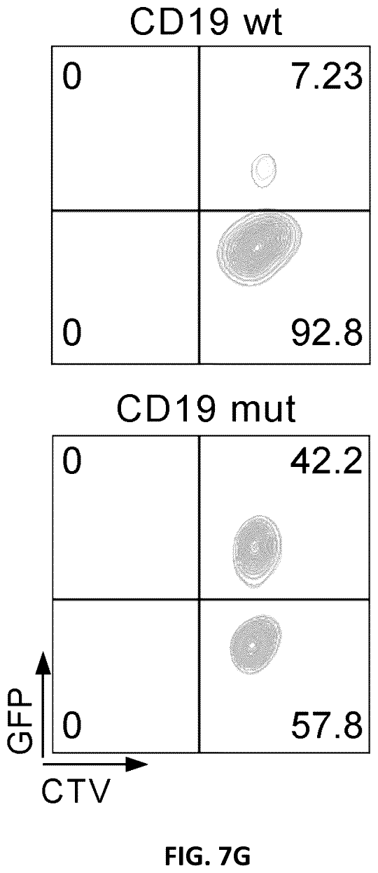

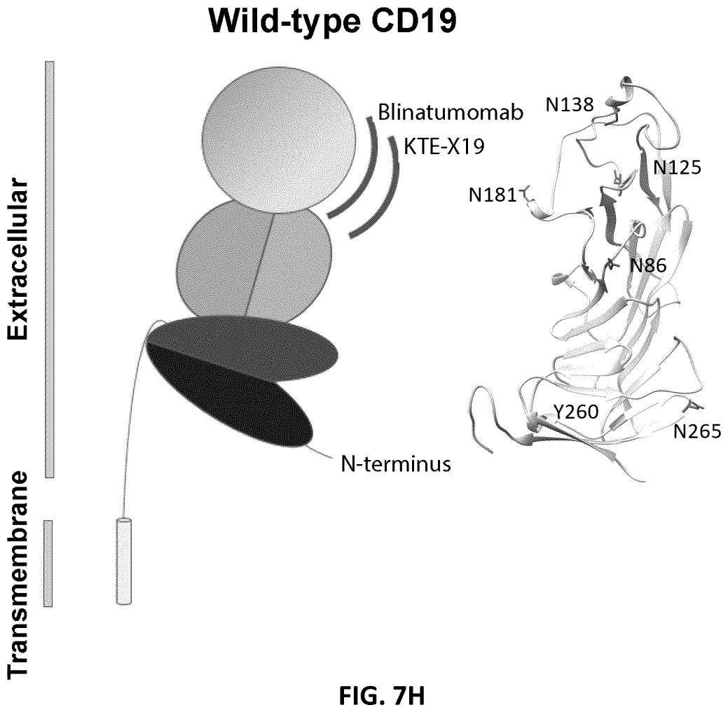

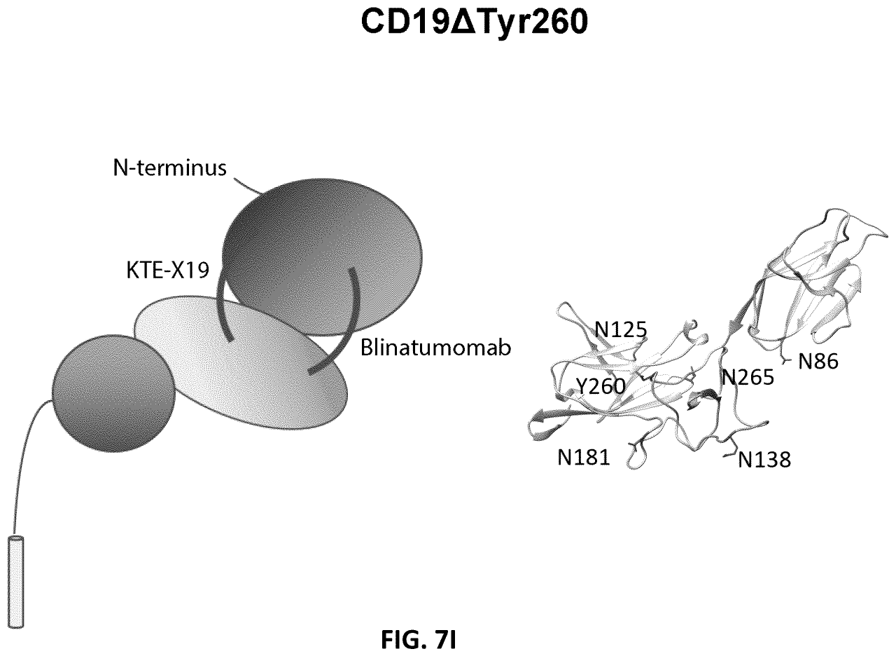

To analyze the effect of CD19ΔTyr260 on the trafficking of CD19 protein to the cell surface, CD19-negative Jurkat cells were transduced with wild-type and Tyr260-mutated CD19 constructs tagged with an internal ribosome entry site (IRES)-driven green fluorescent protein (GFP) reporter (FIG. 7A). Western blot analysis was performed on fractionated lysates of control and transduced Jurkat cells using an anti-CD19 antibody targeting the intracellular protein domain of CD19. Both wild-type and mutant CD19 were detected by western blot in the whole cell lysate, cytoplasmic fraction, and membrane fraction of CD19-transduced Jurkat cells, confirming the transmembrane expression of mutant Tyr260 CD19 (FIG. 7C).

It was confirmed that Tyr260 deletion dramatically alters CD19ΔTyr260 glycosylation (FIG. 7D). Interestingly, two distinct CD19 bands of circa 80-85 kilodaltons (kDa) and 70-75 kDa were observed for CD19 wild-type protein lysates, whereas only the lower molecular weight protein band was observed for the CD19 mutant lysate samples (FIGS. 7C and 7D). After cell lysate deglycosylation, the wild-type and mutant CD19 produced a single CD19-specific protein band with equivalent molecular weights (FIG. 7D). This suggests that the Tyr260-dependent change in molecular weight between wild-type and mutant CD19 protein is mainly due to the dramatic change in glycosylation and is not due to mRNA splicing or post-translational enzymatic protein cleavage.

It was then confirmed that CD19ΔTyr260 makes tumor cells resistant to CD19-specific CAR T-cell killing in vitro at effector to target ratios between 4:1 and 1:1, (FIG. 7E, P<0.0001) and with 1:1 mixing experiments (FIGS. 7D and 7F).

To understand the alteration in glycosylation, in silico models were constructed. Models for CD19 and blinatumomab, as well as their respective docking, were obtained from PDB ID: 6AL5. The model for KTE-X19 was constructed using PDB ID: 6PYC (light chain: 74.27% sequence identity, GQME: 0.79, QMean-1.23; heavy chain: 74.40% sequence identity, GQME: 0.78, QMean: −1.19) with Swiss-Model. A model for CD19ΔTyr260 was constructed with Rosetta and assessed using QMEANDisCo (0.48 versus 0.84 for CD19). Dockings of KTE-X19 to CD19 and CD19ΔTyr260 were performed using a combination of PatchDock, FireDock, and Rosetta (FIGS. 7H and 7I).

In CD19, Tyr260 occurs in the middle of a strand of a 9-stranded beta sheet and links the C-terminus with the N-terminus. At a minimum, deletion of Tyr260 would alter the glycosylation sites at residues 265-268, which occurs at the end of the strand in question. However, the CD19ΔTyr260 model significantly alters the large beta sheet, further disrupting the predicted glycosylation sites and interaction interfaces with KTE-X19 and blinatumomab (FIG. 7I). Interestingly, it has also been shown that mutations proximal to Tyr260 are superfolder mutations, suggesting that this region may be a hotspot for mutations that affect the overall structure of CD19 and its glycosylation sites.

In summary, a novel mutation in CD19 (CD19ΔTyr260) was discovered in a patient with B-ALL that potentially emerged during treatment with blinatumomab and resulted in resistance to blinatumomab and KTE-X19. Although flow cytometry using various reagents did not detect CD19 on the surface of blasts with the Tyr260 mutation and local pathology concluded that pre-KTE-X19 B lymphoblasts were uniformly CD19dim by flow cytometry, it was demonstrated that this mutation is not affecting the trafficking of CD19 to the cell surface. Rather, the mutation results in failure of appropriate CD19 glycosylation, altering the 3-dimensional structure of CD19, resulting in a lack of CD19 recognition by flow cytometry and also resistance to blinatumomab and anti-CD19 CAR T-cell therapy. Additionally, it was discovered that MHC class II genes and their transcription factor CIITA were downregulated post-blinatumomab and post-CAR T-cell therapy, suggesting an additional mechanism of resistance in this patient. The discovery of this CD19 mutation expands the understanding of the mechanisms by which malignant B-ALL cells may gain a clonal advantage in the setting of CD19-directed therapy.

What is claimed is:

1. A method of detecting resistance of a B cell malignancy to an immunotherapy in a subject, the method comprising:
a. detecting a biomarker level in malignant B cells obtained from the subject, the biomarkers selected from a glycosylation state of CD19, an expression or activity of SPPL3, and any combination thereof;
b. comparing the biomarker level detected in the malignant B cells to a reference biomarker level; and
c. determining that the B cell malignancy is resistant to immunotherapy if the biomarker level is substantially different from the reference biomarker level.

2. The method of claim 1, wherein determining that the B cell malignancy is resistant to immunotherapy further comprises determining that the B cell malignancy is resistant to immunotherapy if:
a. the glycosylation state of CD19 is substantially hyperglycosylated or hypoglycosylated compared to the reference glycosylation state in the malignant B cells;
b. the expression or activity of SPPL3 is substantially increased or decreased compared to the reference expression or activity of SPPL3 in the malignant B cells; and
c. any combination thereof.

3. The method of claim 1, wherein the malignant B cells obtained from the subject comprise a mutation affecting CD19 glycosylation.

4. The method of claim 3, wherein the mutation affecting CD19 glycosylation is a CD19ΔTyr260 mutation.

5. The method of claim 1, wherein the malignant B cells obtained from the subject appear negative for CD19 expression as detected via flow cytometry or anti-CD19 antibodies configured to recognize CD19 comprising the reference glycosylation state.

6. A method of treating a B cell malignancy in a subject comprising administering a treatment to the subject comprising one of:
a. administering to the subject a therapeutically effective amount of a CD19-targeted immunotherapy in combination with a therapeutically effective amount of an agent capable of modulating CD19 glycosylation or SPPL3 expression or activity in malignant B cells; or
b. administering to the subject a therapeutically effective amount of a modified CD19-targeted immunotherapy comprising an antibody or a CAR T cell that specifically binds hyperglycosylated or hypoglycosylated CD19 of malignant B cells.

7. The method of claim 6, wherein administering the treatment improves T cell recognition of CD19, increases activation of CAR T cell effector function, or enhances CD19-targeted CAR T anti-tumor cytotoxicity.

8. The method of claim 6, wherein the CD19-targeted immunotherapy comprises a CD19-targeted CAR T cell therapy or a CD19-targeted bispecific T cell engager.

9. The method of claim 6, wherein the B cell malignancy comprises a lymphoma or leukemia.

10. The method of claim 6, wherein the B cell malignancy is recurrent, refractory, or resistant to immunotherapy.

11. A method of selecting a treatment for a B cell malignancy in a subject, the method comprising:
a. detecting a biomarker level of CD19 in malignant B cells obtained from the subject, the biomarkers selected from a glycosylation state of CD19, an expression or activity of SPPL3, and any combination thereof;
b. comparing the biomarker level detected in the malignant B cells to a reference biomarker level; and
c. selecting a treatment for the B cell malignancy based on the comparison of the biomarker level to the reference biomarker level.

12. The method of claim 11, wherein selecting the treatment for the B cell malignancy further comprises:
a. selecting a CD19-targeted immunotherapy for the treatment if the glycosylation state of CD19 is similar to the reference glycosylation state, the expression or activity of SPPL3 is similar to the reference expression or activity of SPPL3, and any combination thereof;
b. selecting one of the CD19-targeted immunotherapy in combination with an agent capable of modulating CD19 glycosylation, or a modified CD19-targeted immunotherapy comprising an antibody, a bispecific T cell engager, or a CAR T cell that specifically binds hyperglycosylated or hypoglycosylated CD19 of malignant B cells if the glycosylation state of CD19 is substantially hyperglycosylated or hypoglycosylated compared to the reference glycosylation state; or
c. selecting for the treatment, the CD19-targeted immunotherapy in combination with an agent capable of modulating expression or activity of SPPL3 if the expression or activity of SPPL3 is substantially increased or decreased compared to the reference expression or activity of SPPL3.

13. The method of claim 12, wherein administering the treatment improves T cell recognition of CD19, increases activation of CAR T cell effector function, or enhances CD19-targeted CAR T anti-tumor cytotoxicity.

14. The method of claim 13, wherein the CD19-targeted immunotherapy comprises a CD19-targeted CAR T cell therapy or a CD19-targeted bispecific T cell engager.

15. The method of claim 11, wherein the B cell malignancy comprises a lymphoma or leukemia.

16. The method of claim 11 wherein the B cell malignancy is recurrent, refractory, or resistant to immunotherapy.

17. The method of claim 11 wherein the malignant B cells obtained from the subject comprise a mutation affecting CD19 glycosylation.

18. The method of claim 17, wherein the mutation affecting CD19 glycosylation is a CD19ΔTyr260 mutation.

19. The method of claim 11, wherein the malignant B cells obtained from the subject appear negative for CD19 expression as detected via flow cytometry or anti-CD19 antibodies configured to recognize CD19 comprising the reference glycosylation state.
